(19) United States
(12) Patent Application Publication
(10) Pub. No.: US 2021/0001157 A1
(43) Pub. Date: Jan. 7, 2021

(54) Personal Protective Face Shield for Preventing Biohazardous, Infectious or Pathological Aerosol Exposure (COVID-19)

(71) Applicants: Tarique Jibril Rashaud, Humble, TX (US); Xavier Jibril Goudeaux, Humble, TX (US)

(72) Inventors: Tarique Jibril Rashaud, Humble, TX (US); Xavier Jibril Goudeaux, Humble, TX (US)

(21) Appl. No.: 16/921,720

(22) Filed: Jul. 6, 2020

Related U.S. Application Data
(60) Provisional application No. 63/048,191, filed on Jul. 5, 2020.

(51) Int. Cl. A62B 17/04; A42B 3/22; A62B 17/00; A61L 9/20; A42B 3/04; A42B 3/30; A62B 18/00

(52) U.S. Cl. CPC A62B 17/04; A42B 3/225; A62B 17/006; A61L 2209/14; A42B 3/0406; A42B 3/30; A62B 18/003; A61L 9/205

(57) ABSTRACT

This novel face shield/window is designed to provide all the protective qualities of traditional OSHA approved PPE face shields. Most critically, however, it also facilitates the creation of a protective Dynamic Ingress Barrier to prevent fine particles and bioaerosols from crossing the face shield plane and transport them away from the often breached and problematic periphery of mask/respirator protective zones. Integral to this process, the novel design enhances a particle's flocculant properties by disrupting the structural integrity of the corona virus electrostatic double layer, and reducing its electrokinetic potential before accelerating it into the airspace. Increasing its ability to flocculate with active viral aerosols, and subsequently settle out of the airspace for cleaning, or captive via a filter.

### TECHNICAL FIELD

The invention described herein relates to Personal Protective Equipment (PPE). More particularly a novel cost-effective, multi-functional personal protective face shield which protects the face and mucus membrane area from infectious, biohazardous, or pathological droplets and, most importantly, aerosols (COVID-19 aerosols). The novelty of this device is its provision of an integral air-manifold and nozzle design which creates a protective dynamic airflow barrier about its periphery. This Dynamic Ingress Barrier (DIB) prevents entry of fine particles, aerosols, viral colloids, and bioaerosols agglomerates (i.e 5 μm range). In addition, this novel design will also deactivate/disinfect, ionize, and accelerate particles and floes to facilitate their peri & orthokinetic flocculation with surrounding viral air panicles for settlement out of work-space air. As depicted in FIG. 30, this device/system will significantly reduce COVID-19 R₍₀₎, by minimizing a specific mode of transmission and addressing the OSHA Hierarchy of Controls on multiple levels.

### BACKGROUND

The current COVID-19 pandemic is transmitting at a historically unprecedented rate and is leaving disastrous global consequences in its wake. Its increased spread has induced heightened attention and efforts in the prevention of severe acute respiratory syndrome coronavirus 2 (SAR-CoV2) from the scientific, medical, and business communities—notwithstanding the general populous. To date, its circulation in Chinn and 94 other countries has resulted in an excess of approximately 200,000 confirmed cases including over 119,000 deaths in the United States alone. Due to R₍₀₎ estimated ranges from 3 to 5.7, and the increasing threat to global health, on Mar. 11, 2020, the World Health Organization (WHO) declared the COVID-19 crisis to be a global public health crisis.

Without question, in the US, the elegance and virulence of this strain have crippled the economy to an extent not witnessed since The Great Depression. Most unfortunately, COVID-19 is having a devastating impact on the lives and livelihoods of American citizens—US hospitals are inundated, front-line health workers are overburdened, PPE supplies are depleted, food supplies are threatened, and a disparate impact has been unleashed upon the nation's most vulnerable communities—including elderly, minority, and those in financially challenged or polluted high-density urban areas. While the transmission of SARS-CoV2 through relatively larger human respiratory droplets (via surfaces etc) and contact with infected persons are clear, the recognition of and concern regarding its aerosol transmission has been elevated exponentially—and justifiably so.

Due to the aforementioned modes of transmission, the CDC produced recommended guidelines for controlling the spread of the deadly SARS-CoV2 virus. A cornerstone of the CDC's guidelines is for the general public to maintain interpersonal social distancing of at least 6 ft. However, while this distancing benchmark may be effective for control of large viral droplet contact transmission, subsequent studies found that viral air clouds contaminated with the COVID-19 aerosols might travel much farther than the 6 feet CDC guideline. These expelled droplets can subsequently be inhaled, land in people's mucus membranes, and deposit onto surfaces where someone can touch them or be resuspended into the air. The aforementioned study published in the Journal of the American Medical Association found that, under the right conditions, liquid droplets from sneezes, coughs, and simple exhalations can travel more than 26 feet and linger in the air for a considerable time—risking someone walking through the cloud and becoming a viral host. The Bourouiba research study focused on these turbulent gas clouds emitted when someone coughs, sneezes, or simply exhales. It was observed that liquid droplets of various sizes drop onto surfaces, while others can be trapped in a cloud that can swirl around a room with a payload of pathogen-bearing fine particulates for a significant amount of time (Bourouiba, 2020). Dr. Stanley Deresinski, a clinical professor of medicine and infectious diseases at Stanford University agrees, “Aerosols are different . . . Very small particles may be suspended in the air for a long time, sometimes for hours. They're suspended by air currents.”. Many studies indicated that aerosols can remain suspended in the air for up to 4 hours and remain oil some surfaces for up to 4 days.

### DETAILED BACKGROUND

The majority of credible literature in this field of study refers to the previously mentioned residual particulates as droplet nuclei or aerosols (Bourouiba, 2020). The most current research indicates that exhalations, sneezes, and coughs consist of both muco-salivary droplets with short-range semi ballistic trajectories and a multiphase turbulent cloud that entrains, traps and carries ambient air clusters of these droplets. Underscoring the fact that breathing, talking, and/or coughing release droplets ranging from submicron to micrometers in size scale.

Descriptively speaking, ‘aerosols’ or bioaerosols will hereafter interchangeably refer to such particles suspended in air and exhibiting Brownian Motion. This agglomeration of bioparticles, presenting varied size profile distributions, prove to be extremely problematic to combat. The graphical illustration in FIG. 31 illustrates that longitudinal research further shows that most literature ill the field utilizes generally accepted particle size and aerodynamic descriptors as follows:

Fine particles of <5 μm in aerodynamic diameter readily penetrate the airways to the alveolar space (Brownian Motion profile); Small panicles of 5-10 μm follow airflow streamlines with the potential capability of both short and long-range transmission; Particles of 10 μm can penetrate below the glottis; Panicles of diameters 10-20 μm, will present hybrid properties of both small and large droplets, but tend to settle more quickly than particles <10 μm; Large droplets of diameters>20 μm refer to those that follow a more ballistic trajectory (i.e. falling mostly under the influence of gravity and can be analyzed via Stokes Law) (Marr, 2020).

Most troubling of all is that bioaerosols also include ‘droplet nuclei’ with aerodynamic diameters of 10 μm or less which can present aerodynamic characteristics more consistent with Brownian Motion as opposed to pure Stokian Physics (Pandis, et al 2006). However, in some situations, where there are strong ambient air cross-flows, for example, even larger droplets can behave acrodynamically similar to aerosols (Tellier et al 2019). As to the virulence of these particles, many would be surprised to learn that research reveals that the majority of viral concentration (RNA copies) is found in the fine bioaerosols and floes<5 μm, as compared to the larger coarse droplets>5 μm (Johnson et al 2011).

Unfortunately, the aerodynamic elegance and size diversity of coronavirus bioaerosols in the nanometer and micrometer range have contributed greatly to its otherworldly virulence (Yan et al 2017). Subsequently, it is this mode of transmission and particle size that inspired Mechanical and Energy Engineers to design Negative Pressure Intubation Rooms in hopes of venting these particles before they can infect front-line hospital personnel (Yan et al 2020). However, such engineering modifications and even the existing traditional Personal Protective Equipment (PPE) designs have proven to be no match for and particularly ineffective at curbing transmission at this molecular/colloidal size scale. For relatively larger particle sizes, research shows that surgical masks may be effective as a direct physical barrier against inhalation into the respiratory tract—If and Only If they do not enter around the sides of the mask. In fact, when scientists were asked about the aforementioned CDC recommendations for the general public to wear commercial masks or other improvised devices, Bourouiba offered a qualified response regarding their efficacy—“Exhalations or violent exhalations such as coughs or sneezes would be deflected to the sides of these masks—as they are not perfectly sealed . . . . It is important, therefore, to understand that such masks are not necessarily protective for the user in terms of preventing inhalation of the residual droplets in the air, which enter from the sides unfiltered . . . (USA Today 2020).

### Design Strategy

Before embarking on a design strategy to minimize the transmission of this aggressive viral strain, the 6-sigma DMAIC methodology was employed to ensure the problem was properly defined and measured in order to increase design effectiveness, rapid licensing and deployment, commercial implementation and ultimately R₍₀₎ control and reduction. Secondly, a Failure Modes Effects Analysis (FMEA) was used, including Pareto Analysis, to identify the failure modes of the existing PPE and focus on eliminating the singular failure mode which contributes to the most failures (80/20 Rule). In parallel, while not directly applicable, because the analysis focus involved various PPE, and other matters within OSHA's purview, a cognizance of Bradford Hill's Causation Criteria was ever-present. Even though the various mask failure modes were considered (i.e. contamination, strap breakage, filter efficiency, tightness of fit, Fit-For-Purpose, among others), it was determined that the elimination of bioaerosol transmission via peripheral mask inward leakage will yield the greatest positive impact in reducing coronavirus R₍₀₎. Specifically, preventing leakage of fine (micrometer/nanometer scale) vitally active particles and agglomerates into the facial mucus membrane area via breaches of mask-to-skin interface and face shield periphery edges. Design and efficacy challenges of the existing art include:

Face Shields—In spite of being used by millions of medical, industrial, and dental personnel, the current art should not be used alone: “Due to the lack of a good facial seal peripherally that can allow for aerosol penetration, face shields should not be used as solitary face/eye protection, but rather as adjunctive to other PPE (protective facemasks, goggles, etc.).” Dr. Raymond J. Roberge National Personal Protective Technology Laboratory, NIOSH, CDC-Pittsburgh.

Surgical Masks—A study by the University of Edinburgh evaluated the protective efficacy of surgical masks and Respiratory Protective Devices (RPDs) against relevant bioaerosols. It was determined that most surgical masks are not certified for use as RPDs as the protection afforded to the user by a surgical mask against infectious aerosols is questionable Other studies show as much as 20% of aerosols in the breath space can leak into the masks.

N95 Respirators—The COVID-19 crisis has proven that legitimate respirators of this type are in short supply. Also, the efficacy of five N95 FFR models was measured using laboratory aerosol as well as poly disperse NaCl aerosols employed for NIOSH particulate respirator certification (TIL tests). The pass rate for 3 of the 5 N95 respirators tested was only 0-5.7% in two independent laboratories combined (TAOH, 2013)

Linen, Gotten, Silk Masks (self-made), etc.—Similar research shows that these masks pass upwards of 70% of fine particles through the mask. Even for the best-crafted cottage mask, other research showed that “Leakages around the mask area can degrade efficiencies by ˜50% or more” (Abbiteja et al, 2020).

The preservation of the citizenry's health and restarting the world economy no longer has to be viewed as mutually exclusive concepts. The aggressive nature of coronavirus transmission calls for a tactfully aggressive solution as an alternative to the currently available passive means of personal protection. To that end, our research indicates that viruses can be removed from indoor air primarily by settling (larger droplets), ventilation (colloids<5 nm), and deactivation (disinfectants, UV, etc). In general, this novel design provides a research-based, data-driven solution to reducing viral transmission by protecting the user as well as those within their work-zone (i.e. surgical teams, meat processors) while combating the virus in each of the aforementioned areas by:
Protecting users from mucous membrane exposure to fine viral particles by providing an ingress barrier to the face area
Providing a means for viral inactivation/deactivation
Increasing ventilation system effectiveness by transporting aerosols away from the head (mucous membrane area) into ventilation airstreams
Increasing particle/colloid settling through induced macro-flocculation for ease of cleaning/sanitation
Increase the effectiveness of N95 respirators, surgical masks, and other personal filtration devices by reducing the percentage of fine aerosol particles at its filter face
Increasing the supply availability of N95 respirators for the medical personnel who need them most by providing an effective alternative to the populous which can be coupled with surgical or homemade masks with equal or greater effectiveness.

This novel face shield/window is designed to provide all the protective qualities of traditional OSHA approved PPE face shields. Most critically, however, it also facilitates the creation of a protective Dynamic Ingress Barrier to prevent fine particles and bioaerosols from crossing the face shield plane and transport them away from the often breached and problematic periphery of mask/respirator protective zones. Integral to this process, the novel design enhances a particle's flocculant properties by disrupting the structural integrity of the coronavirus electrostatic double layer, and reducing its electrokinetic potential before accelerating it into the airspace. Increasing its ability to flocculate with active viral aerosols, and subsequently settle out of the airspace for cleaning, or capture via a filter. When coupled, this novel design increases the protective effectiveness of surgical masks, respirators, and fabric masks by eliminating their primary failure mode of fine particle entry about its periphery due to facial hair, innate porosity, improper donning, or ill-fitting. Most importantly, this design provides a level of redundancy and fail-safe protection for all of the previously stated failure modes. Alone, it will prove to be more effective than surgical masks or home-made masks, and much more deployable and less cost-prohibitive than positive air respirators or full-body PPG products currently available to the general populous, and Industrial workers worldwide.

The scientific crux of the design approach was that of Biomimetics with an undergirding of particle physics, colloidal chemistry, and fluid dynamics among others. This approach was chosen due to the many significant innovations that Biomimetics has sparked over the past two decades through the imitation of models, systems, and elements of nature for the purpose of solving complex human problems. Utilising this framework, key features of the design reflect the following biomimetic systems:

Ingress Barrier—Straits of Gibraltar where the Mediterranean sea meets the Atlantic which presents high levels of differentiation in alien species between the two bodies of water. A hydrologic flow current barrier prevents ingress and mixing of these waters as its impermeability is paramount to the continued existence of the sea-life in each.

Particle Transport A. Ventilation Effectiveness—Anemophilous pollination of various flora types.

Crosswind speeds with sufficient particle entrainment velocities efficiently transport fecundation pollen of aerodynamic sire ranges similar to coronavirus colloids (5 micrometers).

Virus inactivation disinfection—Natural anti-viral and antimicrobial effects of Solar UV in mineral (earth) and aquatic systems.

Zeta-Potential, Flocculation A Settlement—Lakes are often seen to have areas of green algal matter floating on their surfaces. Mimicking natural processes, dissolved air flotation (DAF) is often employed in the removal of this type of material alter coagulation and flocculation (Carty, 2002).

Incorporation of the aforementioned concepts into our design yielded, in general, an integral air-manifold and nozzle design which creates a protective Dynamic Air Ingress Barrier (DIB) that prevents entry of fine particles, aerosols, viral colloids, and bioaerosols agglomerates (i.e 5 μm range). In addition, this novel design will also deactivate/disinfect, ionize, and accelerate particles and floes to facilitate their peri & orthokinetic flocculation with surrounding viral air particles for settlement out of work-space air.

### SUMMARY

A personal protective face shield has two integral and radially bound air chambers which house a plurality of UltraViolet light sources. The inner chamber walls are lined with a photocatalytic film layer which presents a viral deactivating chemical reaction when impinged/absorbed with Ultraviolet light. The air chamber also contains manifolds that direct air to a plurality of removable chambers containing electroceutical fibers capable of inducing molecular level changes to air effluents. The face shield also has a plurality of integral air exit nozzles located about its periphery which can alter effluent fluid dynamic properties.

In accordance with a personal protective face shield design peculiar to the present invention, a remote air moving device can supply airflow to the inlet ports of two integral air plenums. As exemplified m natural solar disinfection, in order to effectively disinfect and deactivate viral air particles after entry into the Anterior Air Manifold Inlet Port, this design utilizes UV-C light and a photoreactive Titania-Silica (TiO2-SiO2) film to deactivate viral particles.

Consistent with another novel aspect of this invention, once the airflow is virally deactivated, and to further destabilize particle structures, the air manifolds direct the air to a flocculation enhancement chamber to lower particle zeta-potential. Fiber within this chamber generates electricity to biomimic the skin's physiologic electrical energy in order to reduce the risk of infection.

Similar to the manipulation of natural properties to facilitate algae removal from lakes, the present design can facilitate various flocculant inducing options based upon desired/avoided energy consumption, pressure drop, and industrial work conditions.

Peculiar to yet another aspect of this novel lace shield design, once the air particle Dow has been virally deactivated, and flocculant enhanced, the manifold then directs airflow to a plurality of nozzles about its periphery. Incorporating the scientific principles seen in the creation of natural flow-current barriers, this exemplary face shield design utilizes incoming pressurized airflow to create Dynamic Ingress Barriers (DIB) of air about the periphery of the face shield to prevent the crossing of any fine viral particles and/or aerosols into the user's breath zone.

Still another novel design aspect of this invention, similar to the natural pollen fecundation transport of crosswinds, the optional nozzles/vane designs are such that particle size and bulk flow velocity is considered to ensure panicle transport with the capture velocity necessary to achieve flocculation and agglomeration with colloids of the mass, size, and aerosol physics within the work environment.

An enabling feature displayed in one embodiment is a temperature sensing adaptive padding with associated digital display. This feature provides visual indication of workers or consumers temperature prior to entering facilities.

A final enabling feature shown in the embodiments herein is voice amplifying device to allow users to communicate clearly without removing the personal protective face shield, and lastly a GPS to be utilized with other features to provide contact tracing data.

These and other objects, features and advantages of the invention will become more apparent from the following description when taken in conjunction with the detailed drawings that show, for purposes of illustration only, the preferred embodiments of the invention.

### BRIER DESCRIPTION OF DRAWINGS

The primary facets of the PPE device can be grasped and understood better with the aid of the following reference drawings and illustrations. Even though the components may not be to granular scale, the novel device, its features, and functions are clearly illustrated.

FIG. 1 is a front perspective view of a personal protective face shield in accordance with a preferred embodiment;

FIG. 2 is a side perspective view highlighting the air plenum, Flocculation Enhancement Chamber, and Posterolateral Nozzle;

FIG. 3 is a rear perspective view highlighting the Posterolateral Nozzles, a voice amplifier, and encased photocatalytic areas;

FIG. 4 is a top perspective view highlighting a plurality of power supplies, a GPS, and plurality of Integrated Circuit Boards;

FIG. 5 is an orthogonal cross-section of an apparatus for a photocatalytic reaction;

FIG. 6 is an illustration of a preferred nozzle design of FIG. 2;

FIG. 7 is a rear view of an adaptive head Padding with embedded tactile thermocouples;

FIG. 8 is a side perspective assembly/exploded view of how adaptive padding attaches to adaptive headpiece.

FIG. 9 is a top orthogonal view of FIG. 8;

FIG. 10 is a general commercial specification for a micro voice amplification apparatus of FIG. 2;

FIG. 11 is an illustration of Bernoulli's Principle applications in Steam Turbine Technology;

FIG. 12 is a front perspective view of an alternative design of FIG. 1;

FIG. 13 is an illustration of an alternative nozzle design of FIG. 12;

FIG. 14 is an illustration of the airflow characteristics of the alternative nozzle design of FIG. 13;

FIG. 15 is a side perspective view of FIG. 12 highlighting an air moving device, central air plenum, alternative Posterolateral nozzle designs, and exiting airflow direction;

FIG. 16 is an illustration of operational Bernoulli principle applications in the alternative nozzle design of FIG. 15;

FIG. 17 is an illustration of a nanoparticles Hydrodynamic Plane of Shear and Zeta-Potential;

FIG. 18 is a commercial specification for Deep UV-LED for FIG. 12;

FIG. 19 is a multiview illustration for 3D-Print manufacture of PLN of FIG. 15

FIG. 20 is an exploded view illustration for the 3D-Print manufacturer of Flocculation Enhancement Chamber of FIG. 2.

FIG. 21 is a perspective illustration of a preferred industrial application of the novel personal protective face shield

FIG. 22 is an optional product design specification for an air moving device of alternative design depicted in FIG. 15.

FIG. 23 is a possible product design specification for a UV-C bulb identified in FIG. 1.

FIG. 24 is a product design specification for a GPS shown in FIG. 4.

FIG. 25 is a product design specification for tactile thermocouples identified in FIG. 7.

FIG. 26. is a possible electrical configuration of voice amplification device shown in FIG. 3.

FIG. 27 is a possible deem cal configuration for a power supply identified in FIG. 4.

FIG. 28 is an optional product design specification for UV blocking acrylic.

FIG. 29 is a product design specification for a digital temperature display device.

FIG. 30 is an illustration of the OSHA Hierarchy of Controls.

FIG. 31 is a graphical description of aerosol particle size and aerodynamic descriptors.

FIG. 32 is an illustration of OPTIX UV-C transmittance protection.

FIG. 33 is on illustration of electroceutical fibers' effect on an air particle's zeta-potential.

FIG. 34 is an illustration of cytopathic effects of electroceutical on coronavirus particles.

### DETAILED DESCRIPTION OF DRAWINGS

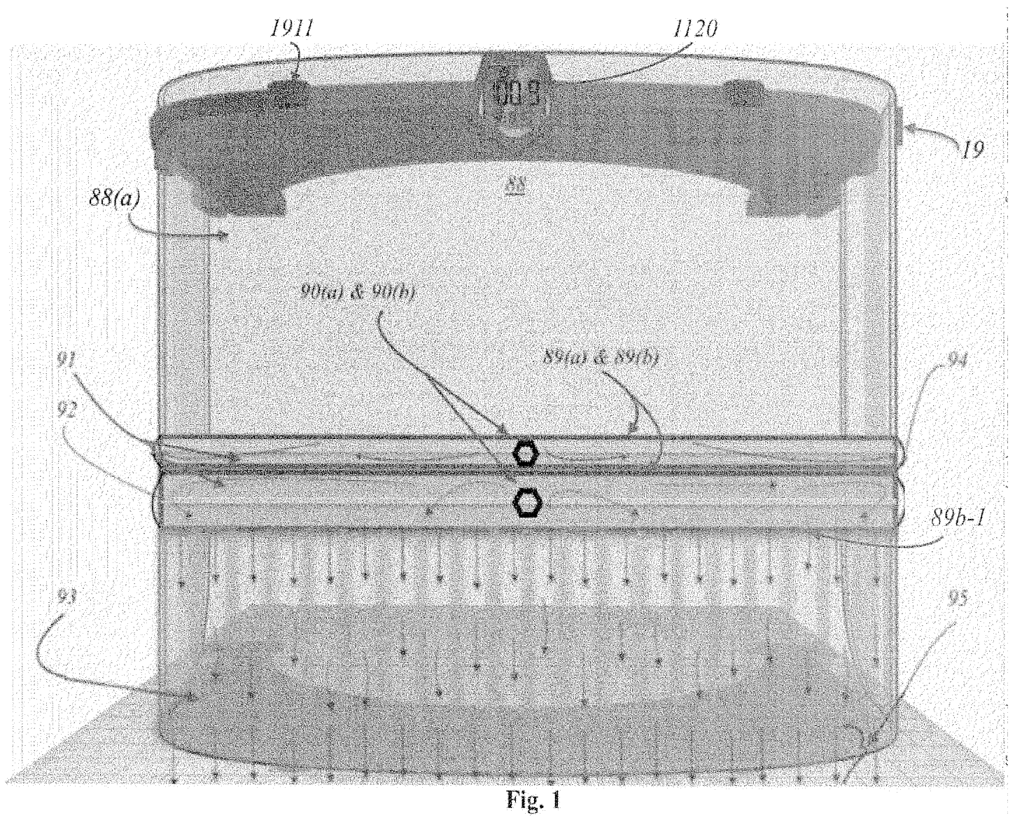

Embodiments of the present invention will henceforth be described in detail with numerical annotations to associated drawings. FIG. 1 is a front perspective view of a personal protective face shield in accordance with a preferred embodiment. As so highlighted in FIG. 1, the face shield is generally composed of one or more air manifold, a plurality of manifold inlets, exit orifices, an cm/off switch, a plurality of nozzles, one or more digital temperature display device, a plurality of UV light-emitting devices;

FIG. 1 shows an exemplary face shield or plastic (polycarbonate) plate window 88 comprising an outer, surface 88a, an inner surface 88b, and geometric edges about its vertical periphery. The face shield 88 design includes an anterior face and two opposing lateral faces. The outer surface 88a has two integral while separate, vertically-centered, and immediately adjacent air manifolds (though covered and concealed by an opaque surface, each is shown herein as clear/translucent for purposes of illustration) 89a and 89b with laterally centered air inlet ports 90a and 90b. The first manifold 89a is integrally designed to be positioned atop a second manifold 89b with each air manifold transversing the width of the anterior face of the face shield 88 and continuing posteriorly with its designed curvature. Air manifolds 89a and 89b each have one or more centrally located UV light-emitting tube 92 which extends transversely for the length of the anterior face. The UV tubes are supported in any conventional fashion to ensure their irradiance is directed onto the catalytic TiO2-SiO2 surface film 91. The inner cavity of each air manifold 89a and 89b is coated circumferentially with a photocatalytic film 91.

Face shield 88 has a plurality of integrally designed air channels 93 which interlace with a plurality of air discharge orifices 89b-1 within the bottom-most edge of air manifold 89b. A digital temperature display device 1120 is located near the uppermost edge of face shield 88 and horizontally centered on outer surface 88a.

The face shield 88 also includes a detachable, relatively lightweight yet durable headpiece adapter 1911. Headpiece adapter 1911 is designed to facilitate attachment to head-gear or a Temperature Sensing Adaptive Headgear foam Padding (T-SAP) for a personal fitting. As exemplified in FIG. 1, headpiece 1911 is coupled to the uppermost periphery of the lace shield 88 via a plurality of inner-to-outer surface, male-female (1911-88) mating interlocks.

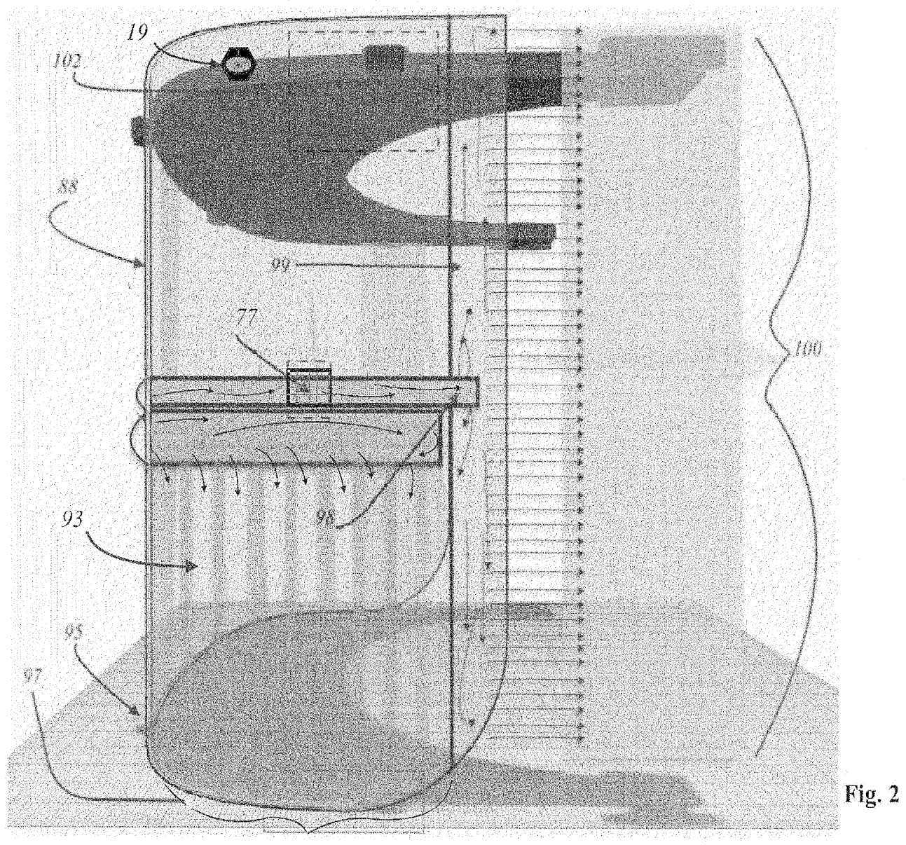

FIG. 2 is a side perspective view highlighting an on/off power switch, a plurality of air plenums, Flocculation Enhancement Chamber (FEC), a posterolateral region. Posterolateral Nozzle, and generated airflow. The face shield 88 has two opposing posterolateral regions 97 with Posterolateral Nozzles (PLN) 99 integrally located at the posterior-most edges of its lateral faces. The PLN 99 has an air inlet port 98 located at its vertical center. The anterior air manifolds 89a and 89b follows the curvature from the anterior face and transverses the lateral face across the posterolateral regions 97. The first manifold 89a terminates at the PLN 99 inlet port 98 while the second manifold 89b transverses the posterolateral region congruently with 89a but terminates just before the junction of the posterolateral region 97 and PLN 99. The positioning of the air channels 93 continues laterally around the curvature of the outer surface 88a from the anterior face and transverses the posterolateral region congruently with 89b and terminates at the junction of the posterolateral region 97- and PLN 99 as well. FIG. 2 also shows the possible location 102 for an optional, in situ, air moving device in lieu of remote air supply. The location of the flocculation enhancement chamber 77. A preferred electroceutical fiber with an activated carbon mesh option 77c is shown in lieu of other FC options described in more detail in a later embodiment FIG. 2 shows a Dynamic Ingress Barrier (DIB) of virally deactivated airflow.

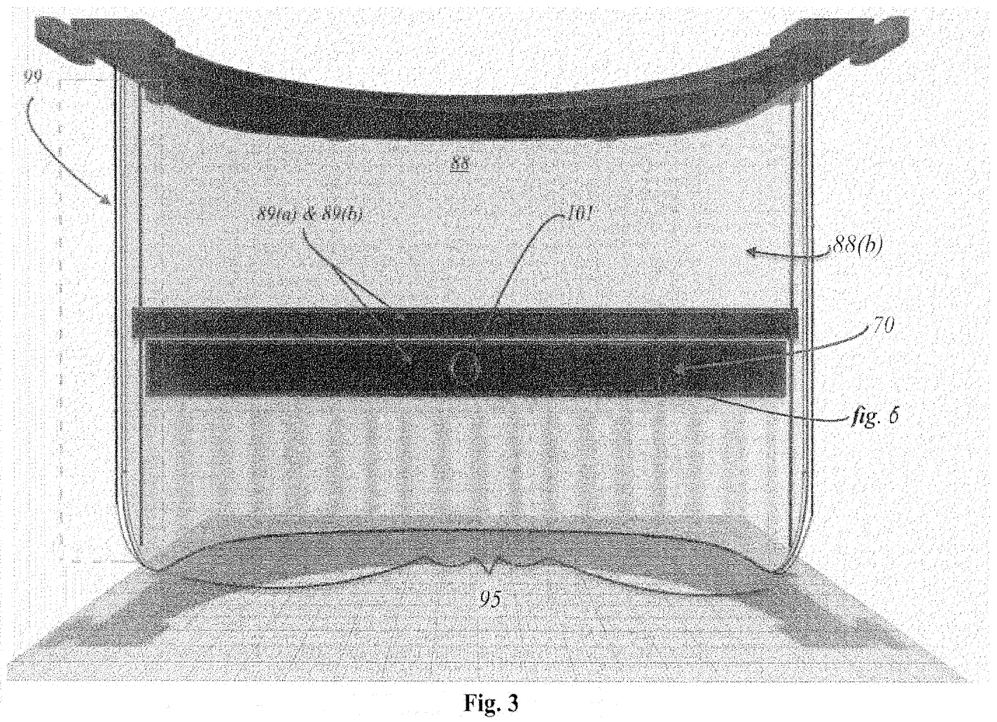

FIG. 3 is a rear perspective view highlighting the inner surface 88b of face shield 88, the posterior faces of 89a and 89b, Posterolateral Nozzles 99, a voice amplification device 101, and photocatalytic area cutaway lines. The face shield 88 includes an inner surface 88b. The acrylic outer layers of air manifold 89a and 89b. The purpose of the acrylic layer is to add an additional layer of protective redundancy to prevent any UV exposure from the photocatalyst UV light of 89a and 89b. A voice amplification device 101 is geometrically centered on the inner surface 88b. The posterior exit face/edge of PLN 99 is also provided for the face shield 88. The airflow zone exits the air channels about the bottom peripheral edges 95 efface shield 88.

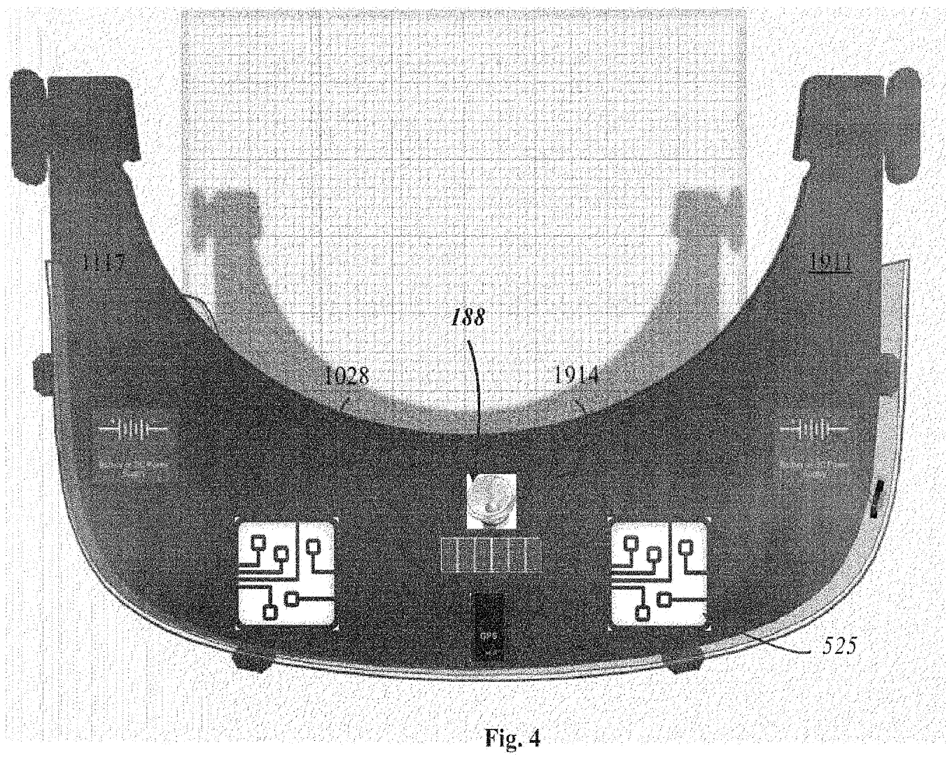

FIG. 4 is a top perspective view highlighting a plurality of power supplies, a GPS, and a plurality of integrated circuit boards. A headpiece adapter 1911 has a plurality of power sources that comprises 1117 and a solar cell 1914. The purpose of the power sources is to supply the required electrical energy for the operation of the GPS 1028 for contact tracing data, voice amplification device 101, UV lube 92a and 92b, optional in situ air blower(s) 102, and a plurality of Integrated Circuit Boards 525.

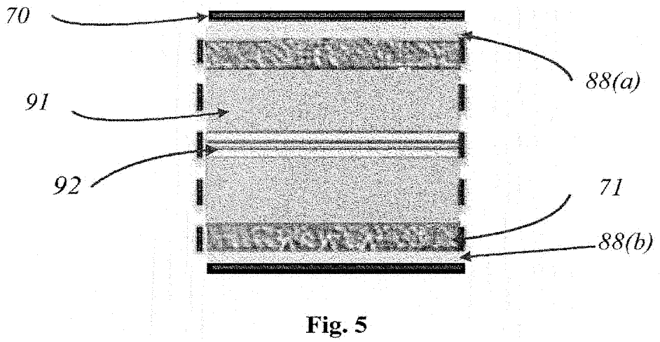

FIG. 5 is an orthogonal schematic of an apparatus for a photocatalytic reaction; A cross-sectional diagrammatic illustration of the primary photocatalytic components of anterior air manifolds 89a and 89b is provided. On the outer surface of manifolds 89a and 89b there is a protective layer of UV filtering acrylic 70 blanketing its polycarbonate substrate to prevent personnel exposure to UV light. The polycarbonate substrate outer surface material is immediately covered by a SiO2 barrier layer 71. The SiO2 barrier layer 71 is stratified by a TiO2-SiO2 photoreactive film layer 91. The polycarbonate inner surface material 88b has protective acrylic layer 70 of equal dimensions and positioning of the anterior air manifolds 89a and 89b to prevent UV light exposure to the user of face shield 88. FIG. 5 lastly shows the UV tube is 92.

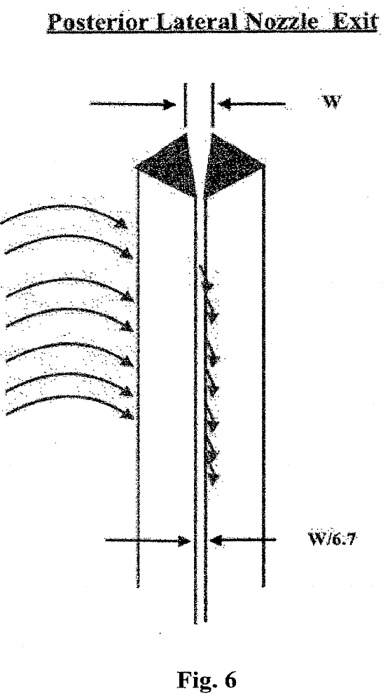

FIG. 6 is an illustration of a preferred nozzle design of PLN 99. The nozzle flow, area is aerodynamically designed with a decreasing area across its flow path.

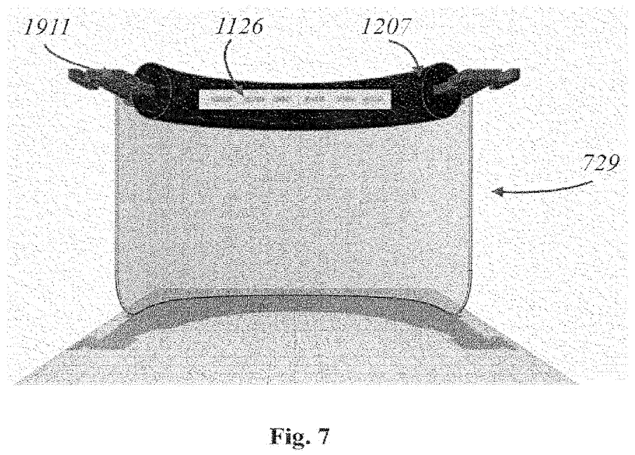

FIG. 7 is a rear view of a face shield device 729, highlighting a Temperature Sensing Adaptive Head Padding (T-SAP) 1207 attached to a headpiece adapter 1911. The T-SAP 1207 is embedded with tactile thermocouples 1126 about its inner surface area.

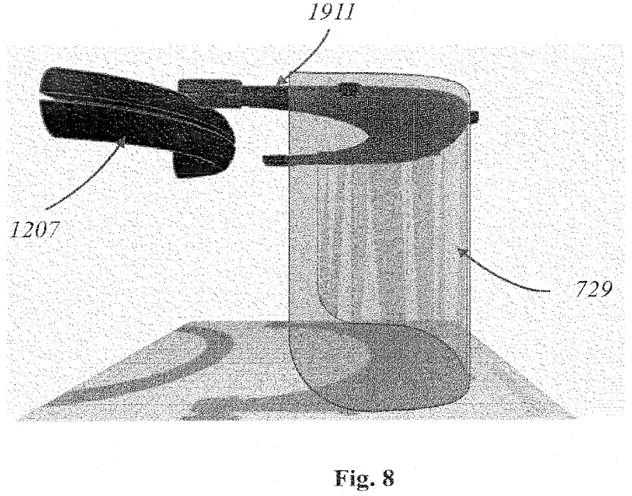

FIG. 8 is a side perspective assembly illustration of T-SAP's 1207 method of attachment to a headpiece adapter 1911 to a face shield device 729, but most importantly how it may attach to face shield 88.

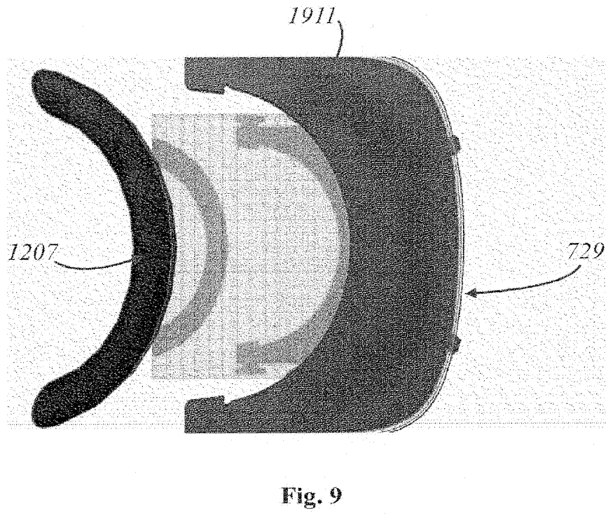

FIG. 9 is a top orthogonal view of FIG. 8; Additional attachment methodology of T-SAP 1207 to a face shield is shown.

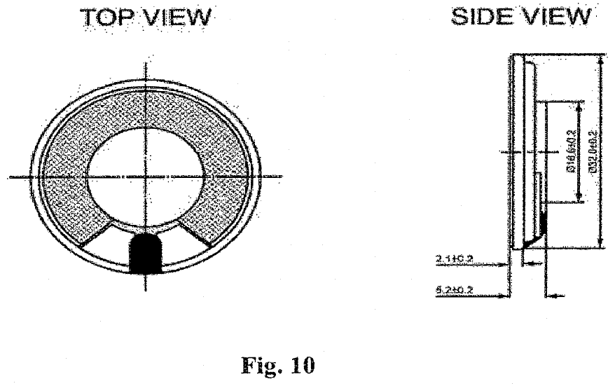

FIG. 10 is a specification for a commercial voice amplification device 101 of FIG. 2.

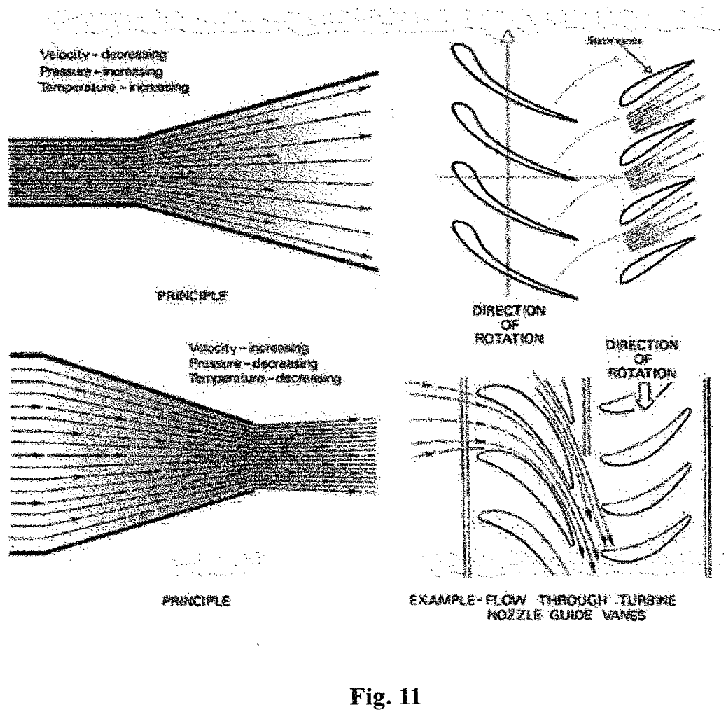

FIG. 11 is an illustration of Bernoulli Principle's application in Steam Turbine Technology; The fluid dynamics of airflow changes as it flows through the nozzle.

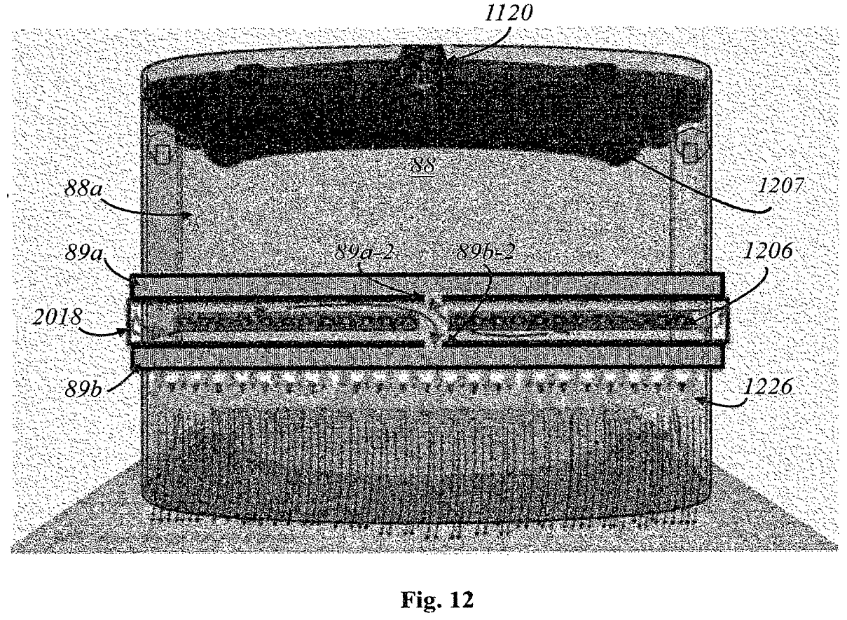

FIG. 12 is a front perspective view of an alternative design of face shield 88 of FIG. 1. The implementation of an alternative nozzle design 1226 in preference to air channel design 93 is shown. The alternatively designed face shield comprises a central anterior air manifold (CAAM) 2018 centrally positioned between the anterior air manifolds 89a and 89b. The CAAM 2018 is separate from, but possessing common walls to the anterior air manifolds 89a and 89b. The anterior air manifolds 89a and 89b have centralized orifices 89a-2 and 89b-2 common to, and in communication with, CAAM 2018. Housed within CAAM 2018 and affixed to the outer surface 88a, is a plurality of alternatively designed Deep-UV LED's 1206 in lieu of the UV tube 92 and excluding any photocatalytic materials of FIG. 5. The incorporated T-SAP 1207 is illustrated in the background and is in electrical communication with digital temperature display device 1120.

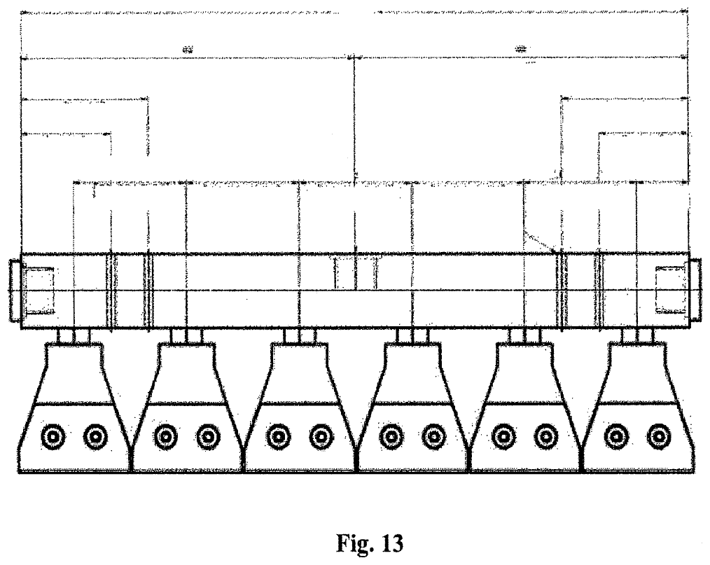

FIG. 13 is an illustration of an alternative nozzle design of FIG. 12. The arrangement of the commercially available alternative nozzle design 1226 is illustrated.

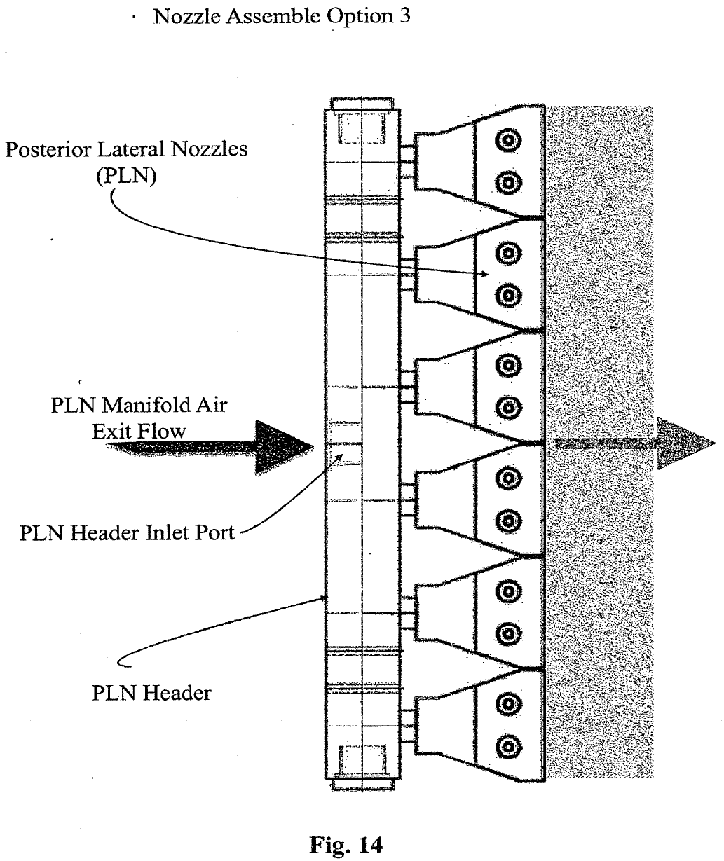

FIG. 14 is an illustration of the airflow characteristics of an alternative nozzle design 1226 of FIG. 12. The alternative nozzle design of FIG. 13 can also be incorporated for use as posterolateral nozzles of FIG. 2 in lieu of PLN 99, based on desired flow characteristics.

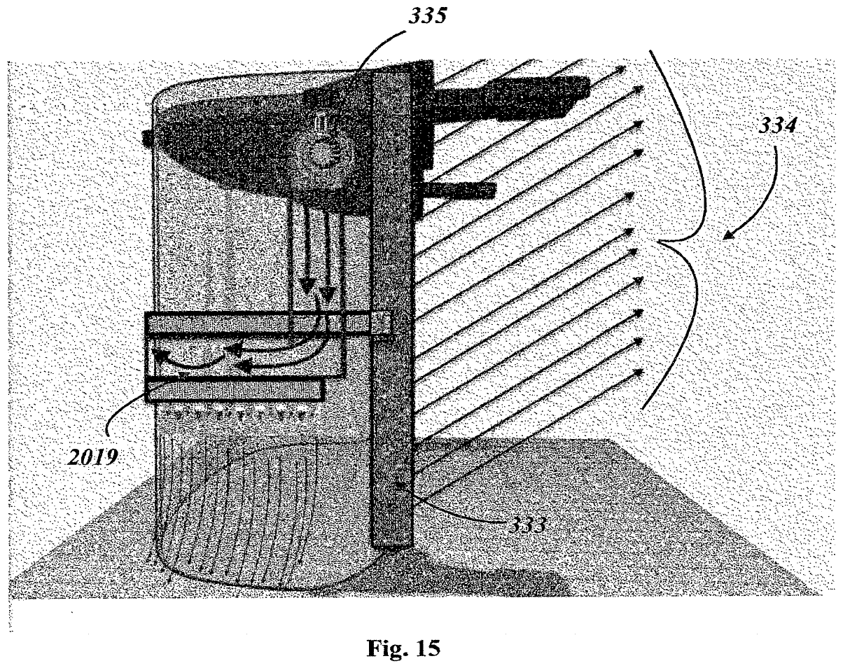

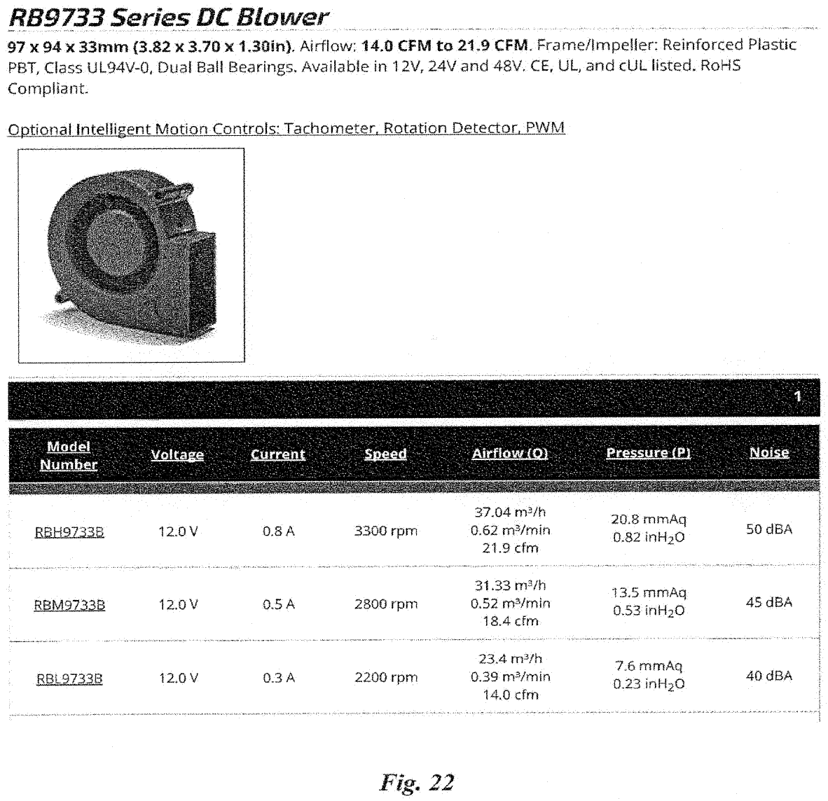

FIG. 15 is a side perspective view of the alternative face shield design 88 of FIG. 12 highlighting an air moving device, a central air supply plenum (CASP) 2019 providing pressurized air to CAAM 2018, yet another alternative posterolateral nozzle design 333, and a directionally exiting airflow characteristic 334. One or more in situ, commercially available micro centrifugal air moving device 335 is located in area position 102. A suitable air moving device for this application can be procured PT Pelonis, Inc. as shown in FIG. 22. The air moving device 335 provides pressurized airflow to CASP 2019 which transverses the width of the posterolateral region of the lateral face of face shield 88 to the CAAM 2018. The resulting DIB created about the bottom periphery of the posterolateral region is illustrated. The directional DIB 334 generated by alternative PLN design 333 is illustrated.

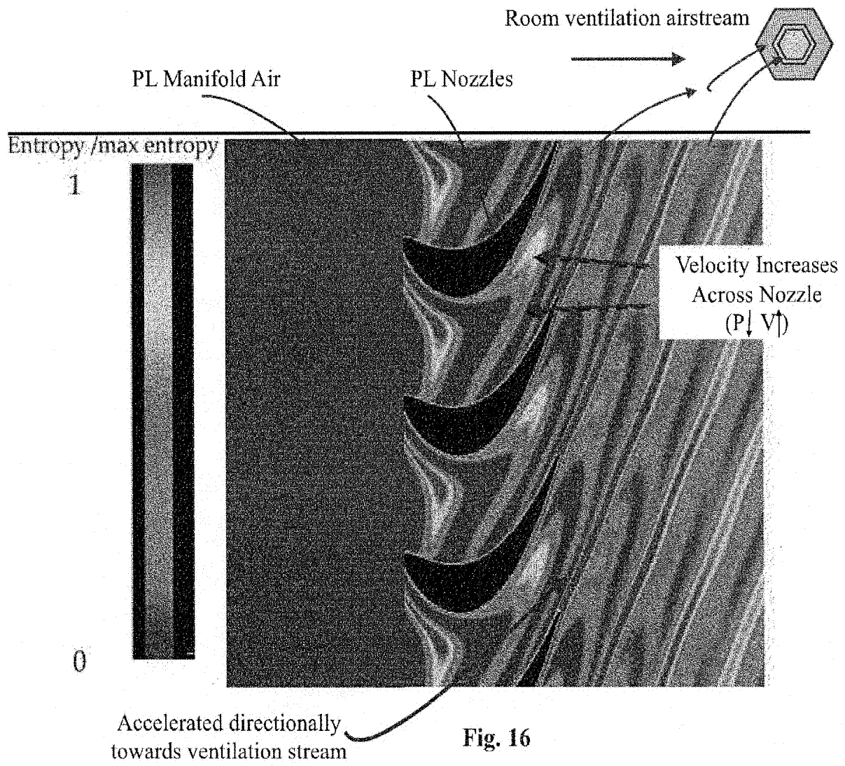

FIG. 16 is an illustration of Bernoulli's Principle in application with the alternative nozzle design of FIG. 15. The specific airflow characteristics generated by alternative nozzle design 333 of FIG. 15 is shown as well as the aerodynamic shape of alternative nozzle design 333 of FIG. 15. Yet another exit flow profile can be achieved optionally to that created by PLN 99 of FIG. 2, based on desired flow characteristics and work environment.

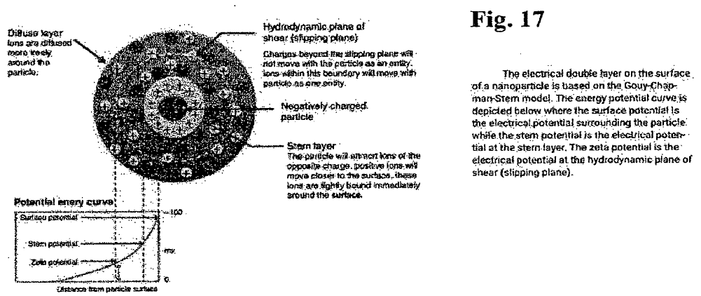

FIG. 17 is an illustration of a nanoparticle's morphology. The nanoparticle has a charged core, an embodying Stem Layer, a Hydrodynamic Plane of Shear, and a Diffuse Ion Layer. The aforementioned particle morphology generates a Surface Potential, Stem Potential, and Zeta-Potential.

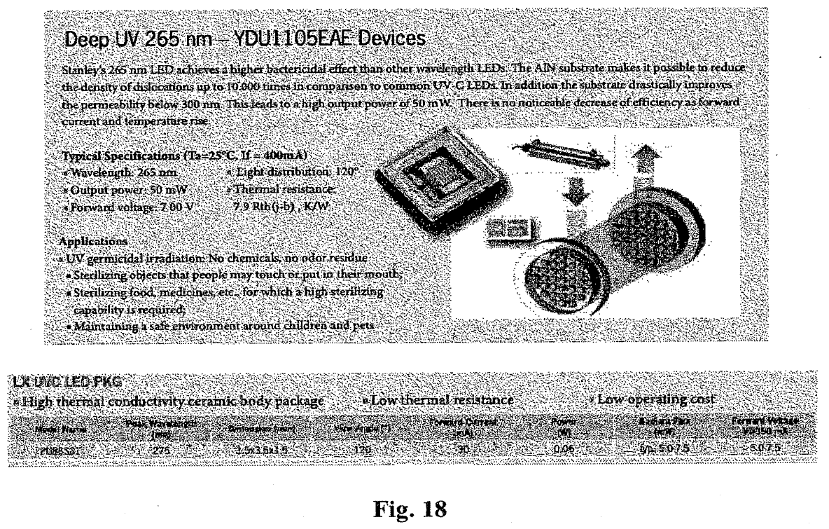

FIG. 18 is a specification for a commercially available Deep-UV LED's 1206 shown in FIG. 12. Specifications for Deep-UV light-emitting diodes 1206 as shown in FIG. 12 for an alternative face shield 88 design. The preferred Deep-UV LEDs described herein can be sourced from LX or Stanely. The typical Wavelength, Output Power, Forward Voltage, Light Distribution, and Thermal Resistance of Deep-UV LED's 1206 manufactured by Stanely is provided. Optionally, shown are similar commercial specifications for Deep-UV LED's manufactured by LX.

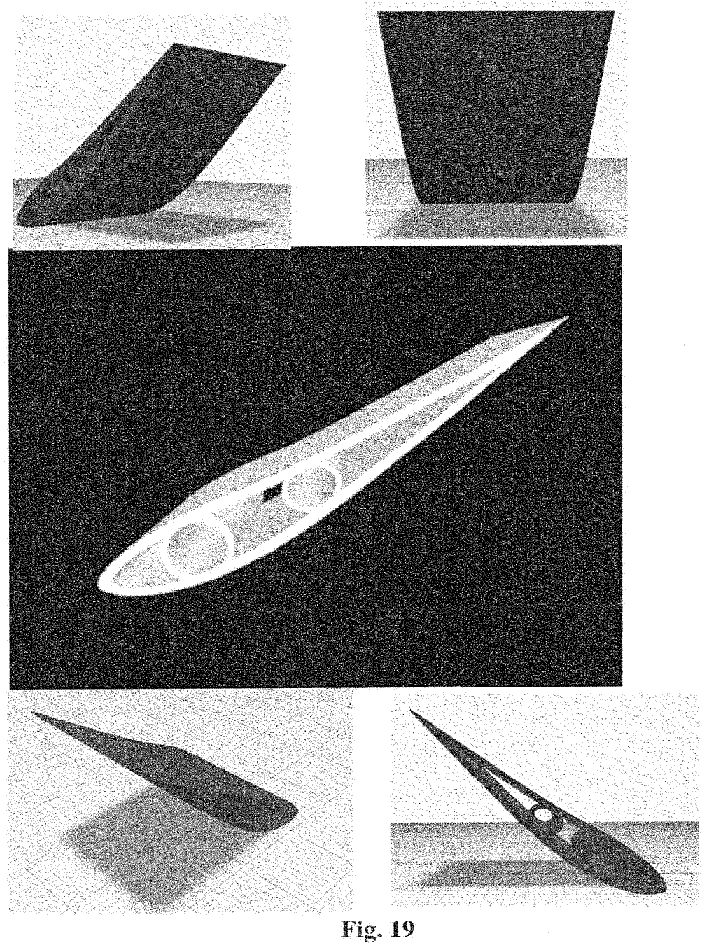

FIG. 19 is a multi view illustration aid to facilitate 3D-Print manufacture of an alternative posterolateral nozzle design by anyone familiar with the art. Various perspective views of the alternative PLN design 333 depicted in FIG. 15 is provided.

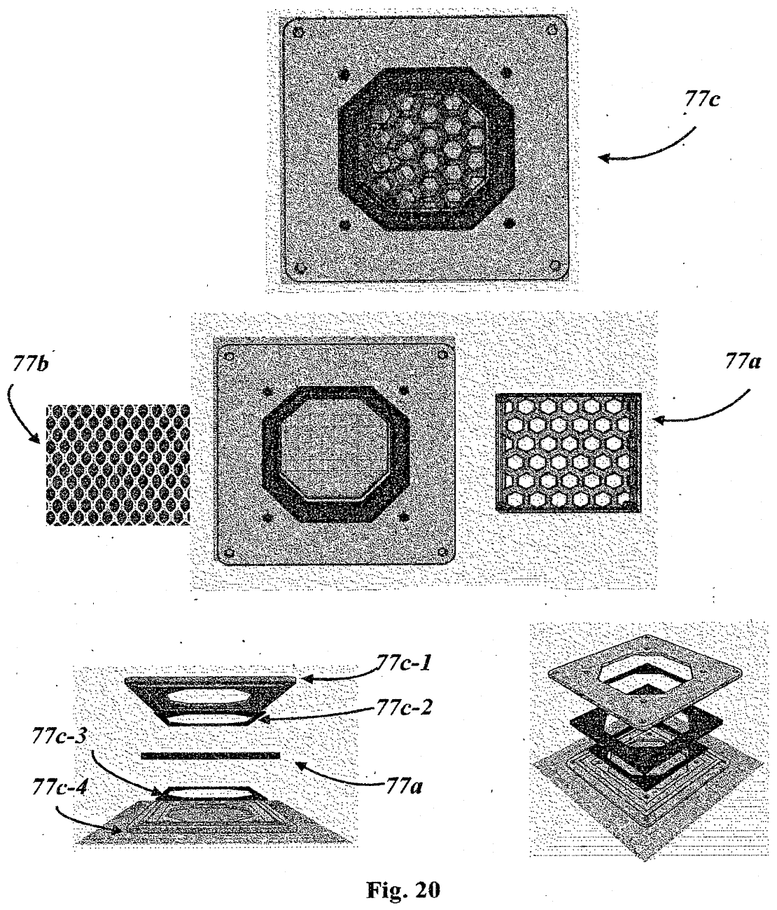

FIG. 20 is an exploded view illustration for the 3D-Prim manufacturer of the Flocculation Enhancement Chamber of FIG. 2. An exploded perspective view of the Flocculation Enhancement Chamber (FEC) 77 of FIG. 2, perspective views of a commercially available electroceutical fiber mesh 77a, an optionally activated carbon filter 77b, and a removable housing assembly 77c1-77c4 is shown. Roth 77a and 77h can be manufactured via 3D printing technology, or specified and purchased commercially.

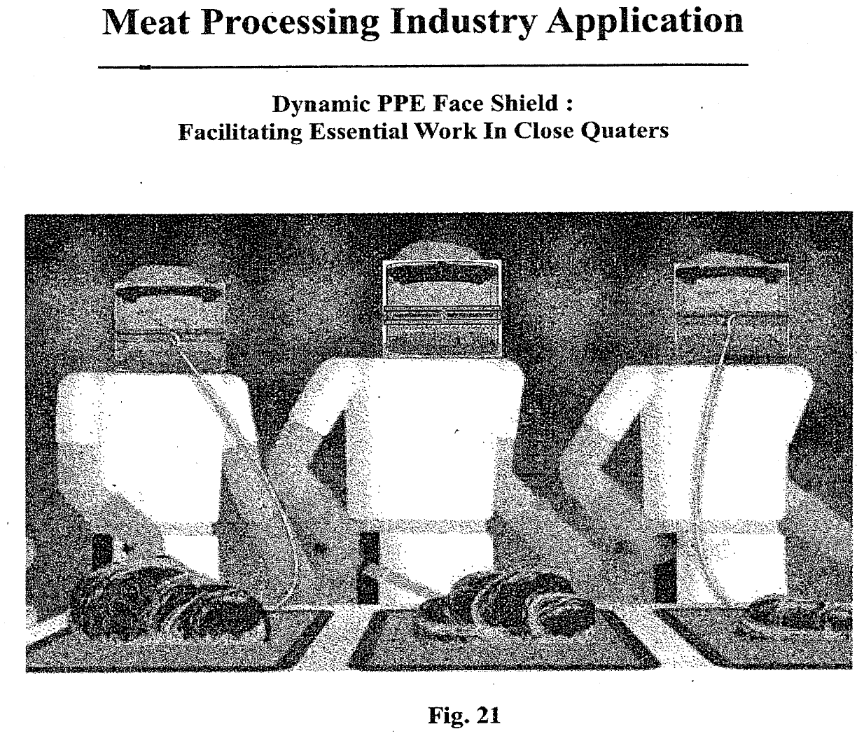

FIG. 21 Illustrates a preferred industrial application of the novel personal protective face shield. The face shield 88 may be applied within a typical work setting in the Meat Processing industry where recommended social distancing is simply not possible or impractical due to the nature of the industrial process. In this industrial application a commercial size centrally located filtered air source would supply pressurized air through a central supply lube affixed longitudinally along the underside of the workstation. Individual air supply branches will connect to the anterior air manifold inlet ports of the protective face shields.

FIG. 22 is a product design specification for an air moving device for an alternative design of face shield 88 depicted in FIG. 15.

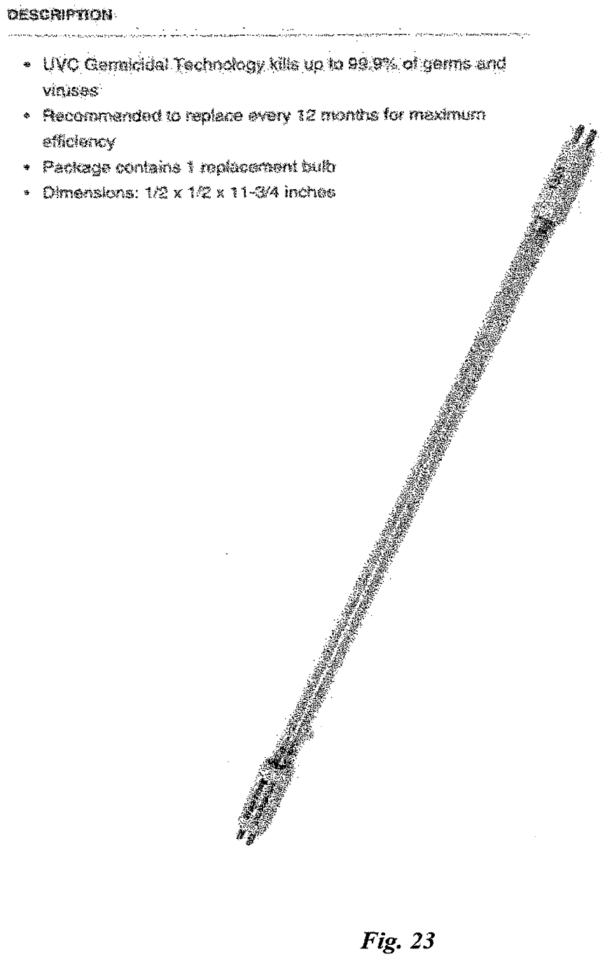

FIG. 23 is a product design specification for a UV-C bulb 92 identified in FIG. 1.

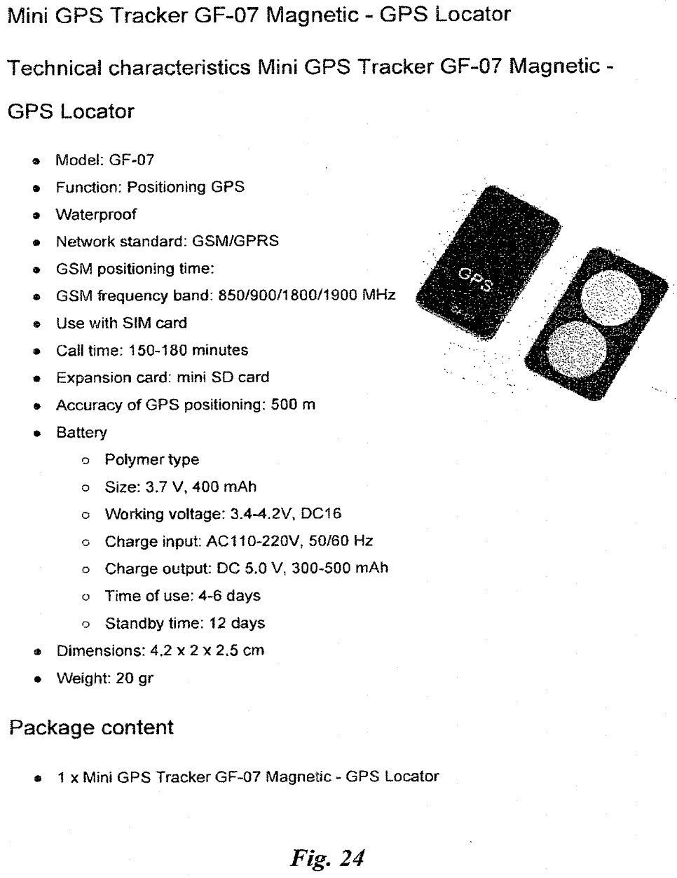

FIG. 24 is a product design specification for a GPS 1028 shown in FIG. 4

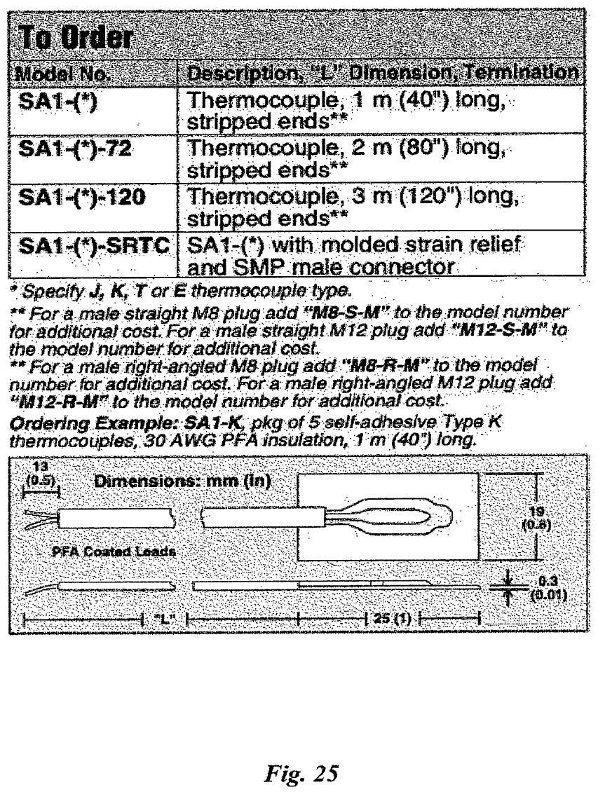

FIG. 25 is a product design specification for tactile thermocouples identified in T-SAP 1207 of FIG. 7.

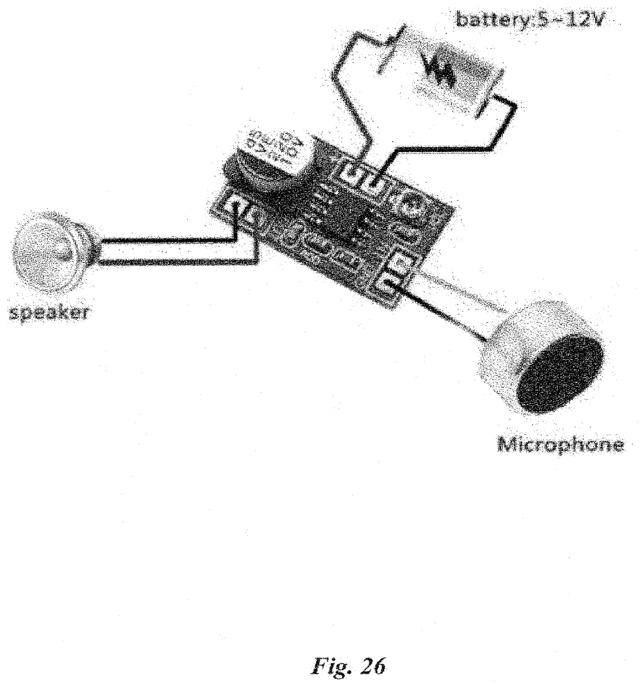

FIG. 26. is a possible electrical configuration for a voice amplification device 101 shown in FIG. 3.

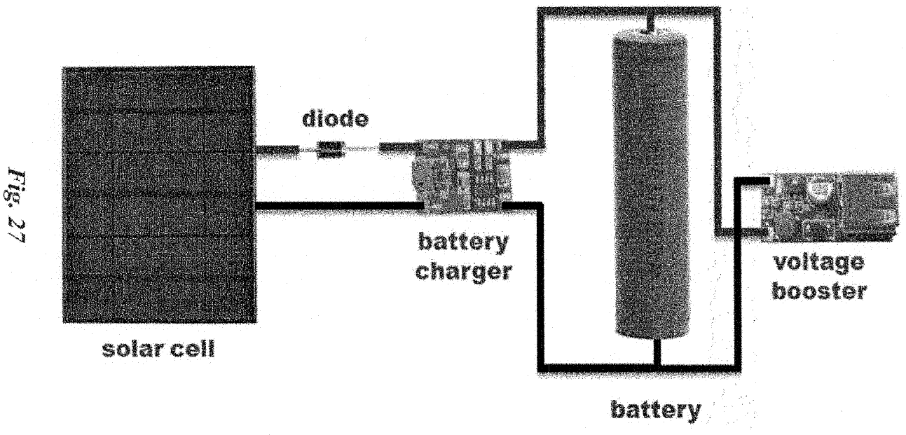

FIG. 27 is a possible electrical configuration for a power supply 1117 identified in FIG. 4.

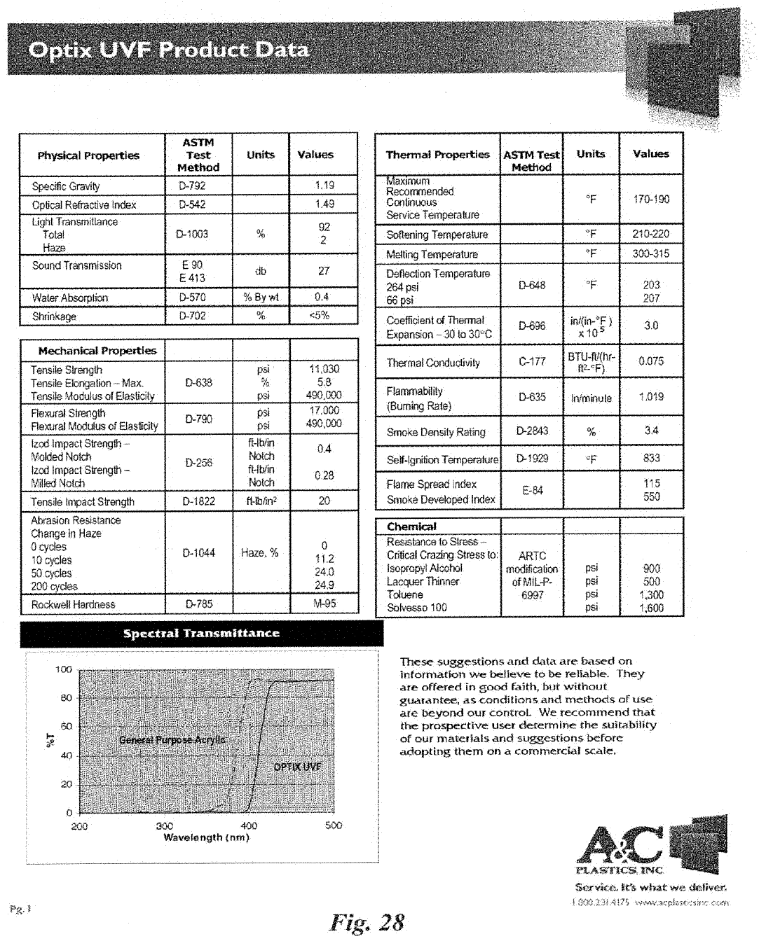

FIG. 28 is a product design specification for UV blocking acrylic 70 of FIG. 5.

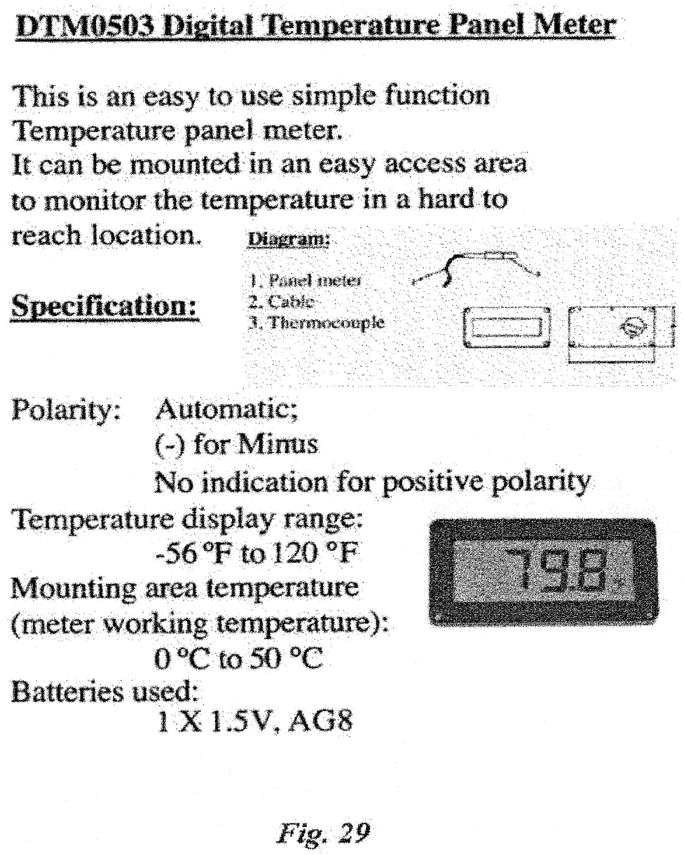

FIG. 29 is a product design specification for a digital temperature display device 1120 of FIG. 1.

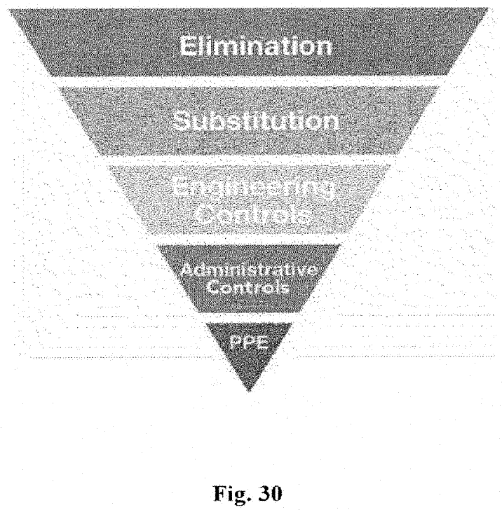

FIG. 30 is an illustration of the OSHA Hierarchy of Controls.

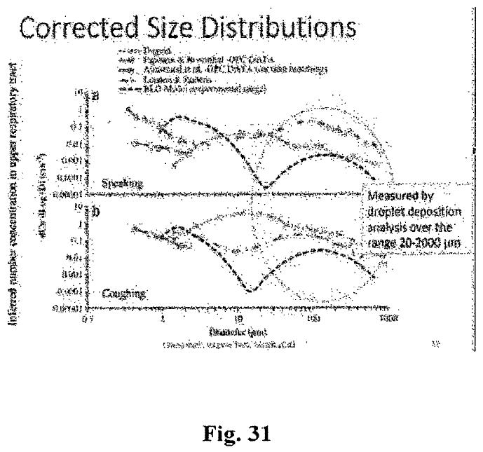

FIG. 31 Graphically illustrates typical exhalation and cough generated aerosol particle sizes and aerodynamic descriptors.

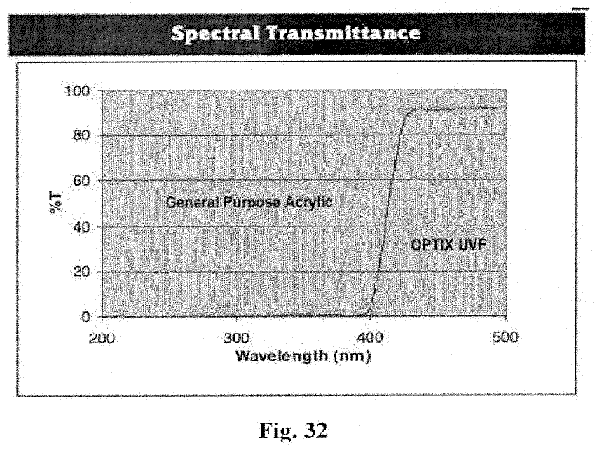

FIG. 32 is an illustration of the acrylic material 70 on the outer surfaces of 89a and the inner surface 88b as shown in FIG. 2, commercially marketed as OPTIX UF for UV-C transmittance protection of the user and surrounding personnel.

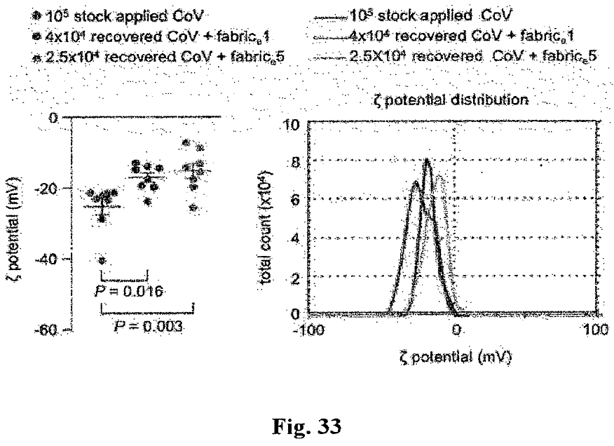

FIG. 33 is an illustration of electroceutical fiber mesh 77a effect on air particle's zeta-potential.

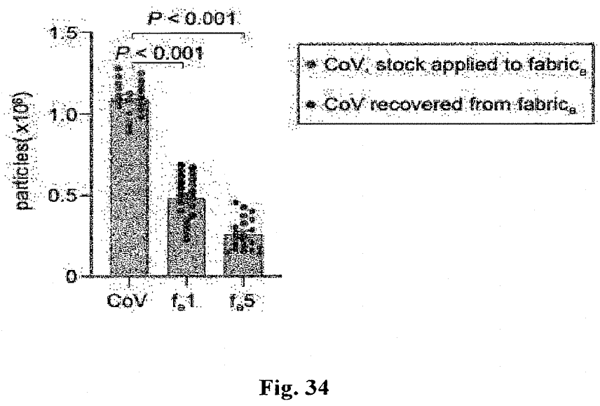

FIG. 34 is an illustration of cytopathic effects of electroceutical fiber mesh 77a on coronavirus particles.

In operation, virally active air is supplied to face shield 88 via a remote portable or stationary air moving device to the Anterior Air Manifolds 89a and 89b. Pressurized air enters manifolds 89a and 89b via air inlet ports 90a and 90b.

To effectively disinfect and deactivate viral air particles after entry into the anterior air manifold inlet ports 89a and 89b, this design utilizes UV-C light and a Titania-Silica film—TiO2-SiO2 91 of FIG. 1 and FIG. 5 to produce a photocatalytic reaction which chemically disrupts and deactivates viral air molecules and destabilizes its' hydrodynamic plane of shear which is identified in FIG. 17 (Mohd et al, 2014). In this novel design, as shown in FIG. 5, UV-C will be generated via a plurality of commercially available UV tubes 92 capable of generating irradiance below 300 lira or in the 250-300 nm range. In an alternative design as depicted in the FIG. 12 embodiment, in order to conserve energy, and optional UV-C light source 1206 in accordance with the research of UC Santa Barbara SSLEEC's can be utilized where Aluminium Gallium Nitride on Silicon Carbide (AlGaN on SIC) Deep-Ultraviolet Light-Emitting Diodes are employed. Such diodes are designed at a preferred irradiance of approximately 278 nm (Burhan et al, 2020). Due to relatively constricted volumetric space, and the close proximity of the air effluent to the photocatalyst within the anterior air manifolds 89a and 89b, as well as the UV intensity of 92 and/or 1206 and the controlled airflow rate, the design conditions are preferential fix thorough deactivation of viral aerosols. Furthermore, to ensure personnel safety, both the user of face shield 88 and surrounding co-workers are protected from harmful UV rays through the application of a shielding acrylic 70 on the outer surfaces of 89a and the inner surface 88h as shown in FIG. 2. The preferred acrylic for this novel design is OPTIX UVF sourced from AC Plastics, Inc. as it provides optimal UV-C transmittance protection as shown in the graph of FIG. 32.

Once virally deactivated in the anterior air manifolds 89a and 89b, the air is directed transversely from the anterior face of 88 in both lateral directions until redirecting posteriorly toward the posterolateral regions 97. As the airflow traverses the width of 97, it enters the flocculation enhancement chamber (FEC) 77 which further destabilizes particle structures. The present design accommodates various in-line FEC 77 options based upon the desired energy consumption, pressure drop, and industrial work conditions. Option 1 (not shown)—An ionizing electrode may be affixed within the air plenums of posterolateral region 97 to ionize flowing air particles prior to their entry into PLN 99 via the dorsal nozzle air supply port 98. This would provide cations or anions which can enhance or diminish particle affinity for agglomeration via Vander Waal Forces and Hamaker forces, it should be noted that the net surface charge of the coronavirus envelope is positive. Option (not shown)—Alternatively, to conserve energy, an in-line bed of compressed beeswax (propolis extract via ethanol or deacetylated Chitosan) impregnated with a high salt composition could be used to dose and destabilize the air particle flow to increase particle flocculency. A chitosan eco-friendly biopolymer with a salt is preferred due to its deacetylation degree variance, dynamic viscosity, hydrophobicity, and Van der Waals interactions, as well as Zeta-potential reduction/particle destabilization properties (Meraz et al, 2016). Option 3) Hie preferred design utilized in this embodiment, and the more sustainable option, incorporates the use of a screen mesh of electroceutical fibers (Polyester+Zn+Ag composition), commercially known as V.Dox and Procellera, as shown in 77a of FIG. 20. The electroceutical properties of the fibers act to depress air particles zeta-potential via the fiber's matrix of embedded microcell batteries which generate minute potential differences of approximately 0.5V which destabilizes the particle's electrokinetic properties as shown in graphs of FIG. 33.

The lowered particle zeta-potential subsequently enhances its chemical flocculence. In complement to the antiviral photocatalytic processes within the anterior air manifolds 89a and 89b, for antiviral design redundancy, the electroceutical fibers further disrupt the cytopathic effects of the virus of coronavirus particles upon contact as illustrated in graph of FIG. 34 (Sen et al, 2020).

It is worth noting the medical utilization of the electroceutical fiber materials described herein have previously been approved by the FDA in the clinical trial (NCT04079998). This described Flocculation Enhancement Chamber (FEC) 77 can be removed for mesh fibers to be analyzed for viral evidence. This enables Air Quality Engineers and EHS personnel to pinpoint specific work areas where dead air spaces of viral aerosols clouds are more present, and equip them to make data-driven engineering modifications to zone-specific work facility ventilation systems. Also, the results of such viral analysis, when coupled with the integral GPS 1028, provide actionable data for Contact Tracing systems (Apple, Google, Kinsa, and others) to identify specific geographic areas of viral presence. It should also be noted that UV-C has been used commercially in HVAC systems and ductwork for many years due to its highly efficient viral and germicidal effectiveness and relatively low risk of ozone. Still, this novel design incorporates a honeycomb design, low pressure-drop, activated charcoal filter 77b within the flocculation enhancement chamber 77c, downstream of the electroceutical fiber mesh 77a for added personnel safety as shown in FIG. 20.

Having been virally deactivated, and flocculaut enhanced, the manifold then directs airflow to a plurality of nozzles about its periphery to create a Dynamic Ingress Barriers (DIB) of disinfected and filtered air 100 of FIG. 2. This is achieved by directing the motive air exiting the air discharge orifices 89b-1 through integral air channels 93 or nozzles 1226 as shown in FIG. 1 and FIG. 12. The DIB 100 is created by the flow of manifold 90b air through PLN 99 or 333 as shown in FIG. 1 and FIG. 12. Similar to the aerodynamic design considerations of Gas Turbine stators vanes and the hydrodynamics of Steam Turbine (Reaction) blades, the nozzles 99 and/or 333 leverages Venturi and Reynolds flow principles among others to provide optimal DIB 100 exit velocity and pressure as illustrated in FIG. 11. Corollary, the novel features allow the flexibility to incorporate nozzle designs capable of inducing either a laminar or a more turbulent flow barrier as dictated by the industrial work environment as in Laser Surgery Rooms, Hospital IntubationRooms, Clean Rooms, etc (Sandlc, 2017). In considering design applications in meat processing industries, as the manifold air travels across nozzle design PLN 99 illustrated in FIG. 6 or alternative nozzle design illustrated in FIG. 14, the aerodynamics of the design creates a decreasing flow area which will cause a decrease in flow pressure and a corresponding increase in laminar flow velocity (V) of the freshly deactivated, electrokinetically dampened exiting particles. The velocity and fluid dynamic properties (critical Reynolds number (Rexer)=5×10⁵ for a flat plate) of the existing air will provide the necessary kinetic energy to help overcome repulsive forces to allow Hamaker influences and collisions with relatively static (Brownian motion) viral air particles. This will encourage the development of macro-flocculation and subsequent settlement out of the air space for cleaning and sanitation. Thereby decreasing the net amount of actively viral air within a given work-area over lime.

A Anal novel design aspect detailed in the embodiments herein, is the optional nozzles/vanes are so designed that particle size and bulk flow velocity is considered to ensure particle transport via adequate capture velocity for the mass, size, and aerosol physics of the targeted aerosols within the work environment. This will be achieved by applying aerodynamic nozzle design factors as illustrated in FIG. 19, in order to manipulate fluid velocity and pressure through design variations of the nozzle flow area as depicted in FIG. 11 and FIG. 14. This feature will ensure user protection by transporting viral aerosols and particles away from the user. In the process, as exemplified in an alternative protective face shield design FIG. 15, the chosen nozzle design of PLN 333 illustrated in FIG. 15 also directs particles toward upper room ventilation airstreams as illustrated in FIG. 16. Thereby increasing the effectiveness of ventilation systems in Intubation, Cleanrooms, or during Laser Surgical Procedures for instance.

### Manufacturing Guidance

The following are examples of general manufacturing methods for the key components identified in the embodiments. All major components of the face shield 88 may be manufactured via 3D printing technology, while complementing functional devices may be procured commercially. Various 3D Printing methods can be employed such as Fused Filament Fabrication (FFF), Fused Deposition Modeling (FDM), Stereolithography (SLA), and others. A multitude of commercial CAD options is readily available to facilitate design modelings such as SolidWorks, Catia, Creo, Autodesk and Dremel, Fusion 360, Rhino, TinkerCad, and others.

### Photocatalytic Reactive Film 91 (Option 1): The Adachi Method

WASH: The polycarbonate (PC) substrate material for 88 should be thoroughly washed with deionized water and ultrasonicated for 10 min to remove contaminants.

PRETREAT: Pretreat regions 89a and 89b of PC substrates by exposing them to oxygen plasma for 1 min to render anchoring hydroxyl groups onto the surface. The oxygen plasma may be created at a pressure=20 Pa, gas flow=10 sccm, and RF power=100 W.

BARRIER LAYER (Pre-Coat): Pre-Coat the pretreated regions 89a and 89b with a SiO2 barrier layer by the dip-coating method, and allow to dry at ambient temperature for 1 hr.

REACTIVE LAYER: The SiO2 precoated PC substrate should then be dip-coated with a TiO2-SiO2 mixture. The preferred TiO2-SiO2 mixture for this embodied design is a 7:3 composition (v/v).

However, depending on case-specific applications, the mixture may be varied to render more advantageous results. The recommended dipping speed should be in the range of ca. 2 mm's, deposition rime of ca. 60 s, and withdrawal speed of ca. 1 mm/s.

DRY: Air dryer at 100° C. for 1 hr while raising the temperature at a rate of 2° C./min.

COATING STABILIZATION: Coat the TiO2/SiO2 coating with a low friction layer of fluoroalkyl silane (FAS) via a simple chemical vapor deposition method. (Adachie et al, 2018).

### Photocatalytic Reactive Film 91 (Option 2): The Sangiorgi 3-D Print Method.

Set-up: 3D scaffolds with an engineered microstructure containing immobilized TiO2 nanoparticles in PLA. Process: Fused Filament Fabrication (FFF) or Fused Deposition Modeling (FDM)

Hardware: Commercial 3D Printer.

Methodology: The Sangiorgi Method (Sangiogi et al, 2019)

Face Shield 88: The figures provided m the embodiments of this document can easily facilitate 3-D print manufacture of the face shield 88 by those versed in the art. For optimal quality, the process should start with high-quality polycarbonate filament, a high-performance desktop 3D printer capable of printing and managing high-temperature materials, and an effective method of bed adhesion to prevent warpage.

Material: engineering-grade high-quality polycarbonate filament.

3D printer: Various models are available with high temp capabilities—preferable printers with a high temp end and bed with an enclosed build chamber (ie Airwolf-3D Axiom Series or similar).

Enclosed Build Chamber: An enclosed print chamber could be utilized to help manage heat and prevent cracking and deformation.

Bed Adhesion: To prevent shrinkage or warp deformations, a heat-activated film can be utilized on the glass work plate which will strongly bond the polycarbonate to the workplace during the print procedure.

Once the print is complete and cooled, most commercial bed adhesion solutions automatically deactivate to facilitate ease of part release from the bed.

### Possible Commercial Models:

Base Model S25A53: Protective Dynamic Ingress Barrier+T-SAP and Digital Body Temperature Display to visibly indicate the users body temperature for screening prior to entering workplaces and places of business+directional nozzle design (upward) (FIG. 16) for increased ventilation system efficiency in Intubation Rooms, Clean Rooms, or Dental Procedures Applications where settlement for cleanup may not be preferable over aerosol venting.
Premium Model S51A1-7 (FIG. 12): Base Model features+UV-CLED on Manifolds for viral Deactivation+GPS to facilitate and provide actionable data for Apple, Google, Kinsa, and other COVID-19 Contact Tracing systems+voice amplifier for ease of communication without PPE removal—possible preferred use by essential needs workers, professional sports teams, etc.
Advanced Model S24A35 (FIG. 1): Premium Model features+Photocatalytic Chamber for enhanced viral deactivation, + flocculant enhancement chamber (3 options).
In situ air supply optional with all models
Integral power source standard with all models
T-SAP with digital temperature display standard with all models.
Solar Rechargeable power source available with Premium and Advanced Models

### Sourcing of Auxiliary Components:

335—Micro-Blower: PTL Pelonis Technologies (https://www.pelonistechnologies.com/)
1206—Deep UV LED: Stanley Electric Co, Ltd. (https://www.stanley-components.com) or LX
91—UV Tube: Hunter Pure Air, Inc.
70—Acrylic: AC Plastics Inc. Optix-UVF Product
77a—Electroceutical Fiber: Vomaris Inc.
1126—Surface Thermocouple with Self-Adhesive Backing: Spectris Inc. (OMEGA.com)
1120—Digital Temperature Display: Circuit specialist Inc. (CircuitSpecialist.com)
1028—GPS: Leak S. L., CIF (Powerplanet.com)
1117—Power Supply & Controllers: Arduino Inc.

It is apparent that innumerable variations of the embodiments described herein before may be utilized. However, these as well as other variations are believed to fall within the spirit and scope of the invention as covered by the claims attached herein.

### REFERENCES

1. Bourouiba, L. (2020). Turbulent Gas Clouds and Respiratory Pathogen Emissions. Jama. doi:10.1001/jama.2020.4756
2. Pandis, S. N., & Seinfeld, J. H. (2006). Atmospheric chemistry and physics: From air pollution to climate change. New York: Wiley. Chapter 9—Dynamics of Single Aerosol Particles
3. Tellier, R., Li, Y., Cowling, B. J., & Tang, J. W. (2019). Recognition of aerosol transmission of infectious agents: A commentary. BMC Infectious Diseases. 19(1). doi: 10.1186/s12879-019-3707-y
4. Johnson et al., 2011, J. Aerosol Sci., https://www.sciencedirect.com/science/article/pii/S0021850211001200 (via Marr, Va. Tech 2020)
5. Yan, J., Grantham, M., Pantelic, J., Mesqoita, P. J., Albert, B., Liu, F., . . . Milton, D. K. (2017). Infectious virus in exhaled breath of symptomatic seasonal influenza cases from a college community. doi:10.1101/194985
6. Total Inward Leakage Measurement of Particulates for N95 Filtering Facepiece Respirators—A Comparison Study. (2013). The Annals of Occupational Hygiene, doi.10.1093/armbyg/mct054
7. Liu, Y., Ning. Z., Chen, Y., Guo, M., Liu. Y., Gab, N. K., . . . Lan, K. (2020). Aerodynamic Characteristics and RNA Concentration of SARS-CoV-2 Aerosol in Wuhan Hospitals during COVID-19 Outbreak. doi:10.1101/2020.03.08.982637
8. Coronavirus might spread much farther than 6 feet in the . . . (n.d.). Retrieved Jun. 11, 2020, from https://www.rnsn.com/en-us/health/medical/coronavirus-might-spread-much-farther-than-6-feet-in-the-air-cdc-says-wear-a-mask-in-public/ar-BB 1297Jm
9. Roberge, R. J. (2016). Face shields for infection control: A review. Journal of Occupational and Environmental Hygiene. 13(4), 235-242. doi:10.1080/15459624.2015.1095302
10. Abhiteja Konda, Abhinav Prakash, Gregory A. Moss, Michael Schmoldt, Gregory D. Grant, and Supratik Guha ACS Nano 2020 14 (5), 6339-6347 DOI: 10.1021/acsnano.0c03252
11. Carty G, O'Leary G. Crowe M (2002). Water Treatment Manuals: Coagulation, Flocculation and Clarification. Washington D. C.: Environmental Protection Agency
12. Mohd Omar, Fateh ah & Aziz, Hamidi Abdul & Stoll, Serge. (2014). Nanoparticle Properties, Behavior. Hate in Aquatic Systems, and Characterization Methods. Journal of Colloid Science and Biotechnology. 3.1-30.10.1166/jcsb.2014.1090.
13. Burhan K. SaifAddin, Abdullah S. Almogbel, Christian J. Zollner, Feng Wu, Bastien Bonef, Michael Iza, Shuji Nakamura, Steven F. DenBaars, and James S. Speck. AlGaN Deep-Ultraviolet Light-Emitting Diodes Grown on SiC Substrates. ACS Photonics 2020 7 (3)554-561 DOI:10.1021/acsphotonics.9b00600
14. Meraz, K. A., Vargas, S. M., Maldonado, J. T., Bravo, J. M., Guzman, M. T., & Maldonado, E. A. (2016). Eco-friendly innovation for nejayote coagulation-flocculation process using chitosan: Evaluation through zeta potential measurements. Chemical Engineering Journal. 284, 536-542. doi: 10.1016/j.cej.2015.09.026
15. Sen A, Khona D, Ghatak S, et al. Electroceutical Fabric Lowers Zeta Potential and Eradicates Coronavirus Infectivity upon Contact. ChemRxiv; 2020. DOI: 10.26434/chemrxiv. 12307214.v1.
16. Adachi, T., Latthc, S. S., Gosavi, S. W., Roy, N., Suzuki, N., Ikari, H., . . . Terashima, C. (2018). Photocatalytic, super hydrophilic, self-cleaning TiO2 coating on cheap, light-weight, flexible polycarbonate substrates. Applied Surface Science, 458.917-923. doi: 10.1016/j.apsuse.2018.07.172
17. Sangiorgi. A., Gonzalez, Z., Ferrandez-Montero, A., Yus, J., Sanchez-Herencia, A. J., Galassi, C., Ferrari, B. (2019). 3D Printing of Photocatalytic Filters Using a Biopolymer to Immobilize TiO2Nanoparticles. Journal of The Electrochemical Society, 166(5). doi: 10.1149/2.0341905jes
18. Miranda, R., Nicu, R., Bobu. E., & Blanco. A. (2016). Efficiency of Chitosan and their Combination with Bentonite as Retention Aids in Papermaking. BioResources, 11(4). doi: 10.15376/biores. 11.4.10448-10468
19. Tim Sandle November 22, By, Sandle, T., & Tim Sandle Dr. Tim Sandle Ph D. (2017, Nov. 17). Distribution of Particles Within the Cleanroom: A Review of Contamination Control Considerations. Retrieved Jun. 30, 2020, from https://www.ivtnetwork.com/article/distribution-particles-within-cleanroom-revicw-coataininati on-control-considerations

The research teams contribution to the described invention is as follows: Turique Jibril Rashaud, BS Mechanical Engineering, Inventor anti Owner of all embodiments and claims herein: Lead research investigations, product concept dr design, engineering analysis, applied biomimetic science, colloidal flocculant design, nano-particle aerodynamics; Xavier Jibril Goudeaux, Undergraduate Neuroscience Pre-Med (Co-Inventor): Longitudinal research of COVID-19 literature on virulence, incident rate, prevalence, molecular structure & zeta-potential, S-protein RNA de-activation. Curating OSHA, NIOSH, and CDC PPE criteria; Design Review for the feasibility of 30-Print Manufacture via Auto-Desk A Dremel software or other FFF/FDM technologies. Ashanti Goudeaux Williams MBA, BS Biology: Advisement on Biofuel Technology knowledge-transfer; De'Rius Rashad Goudeaux MS Occupational Safety & Health Environmental Mgmt.: Advisement on OSHA/NIOSH standards, 6-Sigma and Bradford Hill's Causation Criteria.

1. A personal protective face shield operable to protect the users entire face or portions thereof, or mucus membranes from exposure to or impacts from flying fragments, objects, large chips, liquids, fluids, particulates; inhalation of infectious, biohazardous, or pathological aerosols, bioaerosols, or airborne viral contagions by creating a dynamic ingress barrier of tangentially flowing air;
A previously described personal protective face shield which also deactivates viral air particles and alters their chemical and morphological characteristics to enhance their ability to flocculate with colloids and agglomerates in the surrounding environment to induce their settlement out of the air;
A previously described personal protective face shield which also transports aerosols and/or line air particles away from the face, mucus membrane and head area into room ventilation airstreams; and
A previously described personal protective face shield which increases the efficacy of N95 respirators, surgical masks, and other personal filtration devices by reducing the concentration of fine aerosol particles at their filter face and periphery.

2. The personal protective face shield of claim 1, comprising: a transparent, molded, shaped or 3D-printed protective screen; a modestly pliable headpiece adapter coupled to said protective screen; one or more air manifold; one or more air inlet port or orifice; one or more air exit orifice or port; a plurality of air channels; one or more air exit nozzle; one or more UV light-emitting device; one or more Flocculency Enhancement Chamber.

3. The personal protective face shield of claim 1, wherein said protective screen is of multi layer polycarbonate material.

4. The personal protective face shield of claim 1, wherein said headpiece adapter supports a plurality of power sources, control boards, integrated circuits and an assortment of connective wiring.

5. The personal protective free shield of claim 1, wherein said air manifold(s) is integral or attached to the protective screen.

6. The personal protective free shield of claim 1 wherein the inner cavity of said manifold(s) is lined with multiple layers of photocatalytic material for the purpose of producing a photocatalytic reaction.

7. The personal protective face shield of claim 1, wherein said photocatalytic layers of the air manifold(s) contains photocatalytic materials such as TiO2 and TiO2-SiO2 due to its photocatalytic reactivity and self-wetting properties.

8. The personal protective face shield of claim 1, wherein said air manifolds house one or more Ultra Light emitting (U V) source for the purpose of emitting and/or irradiating Ultraviolet tight onto said photocatalytic materials to induce a photocatalytic reaction.

9. The personal protective lace shield of claim 1, wherein said UV source emits at wavelengths in the 250 nm to 300 nm UV-C range.

10. The personal protective lace shield of claim 1, wherein said air manifold(s) contains one or more permanently fixed or removable Flocculency Enhancement Chamber (FEC) downstream of the air manifold inlet ports.

11. The personal protective face shield of claim 1, wherein said FEC contains at least one or more material or process capable of lowering the zeta-potential of particles within effluent air.

12. The personal protective face shield of claim 1, wherein one of the said material or processes chemically capable of lowering the zeta-potential of effluent air is an electroceutical fiber.

13. The personal protective face shield of claim 1, wherein said electroceutical fiber within the FEC generates electricity to biomimic the human skin's physiologic electrical energy used to reduce the risk of infection.

14. The personal protective face shield of claim 1, wherein said electroceutical fiber is sold under the commercial name of REDOX or others, and is comprised of a matrix of moisture-activated silver (AG) and Zinc (Zn) composed microcell batteries embedded in its substrate and undergoes a chemical REDOX reaction when its surface is exposed to moisture.

15. The personal protective face shield of claim 1, wherein said FEC contains one or more activated carbon filter to filter any residual ozone created during the photocatalytic reaction.

16. The personal protective face shield of claim 1, wherein said electroceutical fibers are removable to facilitate viral analysis by EHS, CDC, OSHA, NIOSH or others.

17. The personal protective face shield of claim 1, wherein said air manifold (s) have one or more inlet port located on its surface to accept, direct and distribute motive air along the surface of said protective screen.

18. The personal protective face shield of claim 1, wherein said air manifold(s) has a plurality of exit orifices to direct air through air channels.

19. The personal protective face shield of claim 1, wherein said air channels are integral or affixed, and direct air from said air manifold(s) to air exit orifices located on the periphery of face shield to create a dynamic ingress barrier of tangentially flowing air as it exits.

20. The personal protective face shield of claim 1, wherein said protective screen contains one or more nozzle or nozzle assembly on or near its periphery so designed as to alter the effluent airs fluid dynamics and aerodynamic particle properties.

21. The personal protective face shield of claim 1, wherein said exiting effluent air flows tangentially out of the periphery of the face shield to create a dynamic ingress barrier of air.

22. The personal protective face shield of claim 1, further comprising: a least one Temperature Sensing Adaptive Padding (T-SAP), one or more global positioning system device (GPS), an on/off switch, one or more digital temperature display device, one or more voice amplification system, a plurality of power sources including a solar cell, and a plurality of optional air moving devices.

23. The personal protective face shield of claim 1, wherein said T-SAP is so designed as to attach to the said headpiece adapter.

24. The personal protective face shield of claim 1, wherein said T-SAP has one or more tactile sensing thermocouple secured adhesively to its surface for sensing the users body temperature at the forehead interfaces.

25. The personal protective face shield of claim 1, wherein said T-SAP is in electrical communication with a temperature display device via a plurality of electrical wires and integrated circuits in order to visibly display the users body temperature for screening prior to entering workplaces and places of business.

26. The personal protective face shield of claim 1, wherein said GPS is used in conjunction with viral analysis of FEC electroceutical fibers to facilitate contact tracing and viral activity tracking.

27. The personal protective face shield of claim 1, wherein said GPS and FEC viral analysis are coupled to provide actionable data to national contact tracing networks and systems.

28. The personal protective face shield of claim 1, wherein said voice amplification system has a microphone adhesively attached to the inner face of the face shield, and a speaker device attached to the adaptive headpiece

29. The personal protective face shield of claim 1, wherein said power supplies provide the necessary energy to operate the apparatus.

30. The personal protective face shield of claim 1, wherein said solar cell is electrically connected via wiring to a rechargeable power source through an Arduino integrated circuit and controller.
